(19) United States
(12) Patent Application Publication
(10) Pub. No.: US 2026/0283205 A1
(43) Pub. Date: Sep. 24, 2026

(54) ACYLATED DIHYDROCHALCONES, METHODS OF PRODUCTION AND USES THEREOF

(71) Applicant: SYMRISE AG, Holzminden (DE)

(72) Inventors: Lukas Konstantin SANDFORTH, Holzminden (DE); Bastian ZIRPEL, Holzminden (DE); Jakob Peter LEY, Holzminden (DE); Thomas RIESS, Holzminden (DE); Tom SOMERS, CC Ede (Gld) (NL); Hans-Ulrich HUMPF, Münster (DE)

(73) Assignee: SYMRISE AG, Holzminden (DE)

(21) Appl. No.: 19/167,995

(22) PCT Filed: Apr. 11, 2023
(86) PCT No.: PCT/EP2023/059412

§ 371 Date: Sep. 23, 2025

(51) Int. Cl. A23L 27/30; A23L 27/00; C07H 1/00; C07H 15/203; C12P 19/44

(52) U.S. Cl. CPC A23L 27/33; A23L 27/36; A23L 27/88; C07H 1/00; C07H 15/203; C12P 19/44

(57) ABSTRACT

The present invention relates to the use of a compound or a mixture of two or more compounds of formula (I) for providing a sweet taste and/or modulating the sweet taste of one or more sweet tasting substance(s) and to a method for producing such a compound. Further, the present invention relates to a compound of formula (I), to a composition comprising such a compound and to a method for modulating the sweet taste of one or more sweet tasting substance(s).

The present invention relates to the use of a compound or a mixture of two or more compounds of formula (I) for providing a sweet taste and/or modulating the sweet taste of one or more sweet tasting substance(s) and to a method for producing such a compound. Further, the present invention relates to a compound of formula (I), to a composition comprising such a compound and to a method for modulating the sweet taste of one or more sweet tasting substance(s).

The sweet taste of food and beverages containing considerable quantities of sugar is often perceived as desirable by customers. However, there is a growing awareness that sugar, such as sucrose, glucose and fructose when consumed in significant quantities has a detrimental effect on customer's health. Weight gain and associated cardio-vascular issues, insulin resistance and type 2 diabetes as well as oral health problems are among the potential negative impacts of a high sugar diet.

Sweet modulating taste solutions are an important tool to reduce the sugar content in sweetened foods by maintaining the overall taste profile. Existing solutions used in the flavor industry like advantame, neohesperedine dihydrochalcone, rebaudiosides, rubusosides, mogrosides and erythritol have their own disadvantages such as a negative aftertaste, bitter taste aspects, low sweetness modulating potency and the fact that some of the compounds are provided as synthetic, non-natural compounds.

Stevia rebaudiana based extracts and isolates are widely used among Rubus suavissimus and Siraitia grosvenorii improve the sweet taste. Such natural sweet modulating systems are preferred solutions for product developers nowadays. However, sweet modulating terpene glycosides such as rebaudiosides, rubusosides and mogrosides possess inherent non-sugar like taste attributes, as described above (such as a negative aftertaste and bitterness). Dihydrochalcones are another class of sweet compounds such as neohesperidine dihydrochalcone which is, however, an artificial compound.

In contrast, its derhamnosylated form hesperetin dihydrochalcone-4′-O-β-d-glucoside (HG) represents a natural dihydrochalcone as it has been identified in natural source material (Balanophora harlandii) (Prakash et al., 2020).

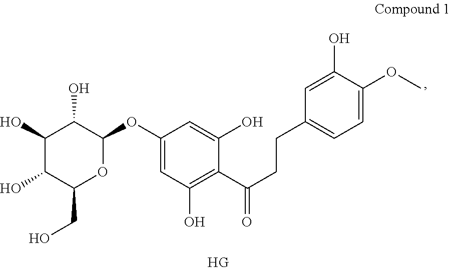 [SMILES: COc1ccc(CCC(=O)c2c(O)cc(O[C@H]3O[C@@H](CO)[C@H](O)[C@@H](O)[C@@H]3O)cc2O)cc1O]

HG has been described as a sweet modulating compound in U.S. Pat. No. 10,463,063 B2, which discloses that 5 mg/kg HG in water does not taste sweet, 10 mg/kg HG is isosweet compared to 0.5% sucrose and 20 mg/kg HG is isosweet compared to 1% sucrose solution. Furthermore, application of HG is also described in sweetener blends such as in US 2022/0295833 A1.

HG can be obtained from the artificial compound neohesperidine dihydrochalcone via fermentation as described in CN 114181987 A, via enzymatic derhamnosylation as described in CN 101787062 A or from hesperidin via derhamnosylation and hydrogenation using a metal catalyst as described in CN 108220366 A.

However, as described above, there is a large need for new and improved sweet tasting and/or sweetness modulating compounds.

It was thus a primary objective of the present invention to provide compounds providing a sweet taste, preferably an improved sweet taste, and/or modulating the sweet taste of one or more sweet tasting substance(s) including sweeteners. Thus, an objective of the present invention was to facilitate reducing the amount of sugar, sweet tasting substances or sweeteners in a food or beverage while maintaining the overall sweetness.

The primary objective of the invention is solved by the use of a compound or a mixture of two or more compounds of formula (I)

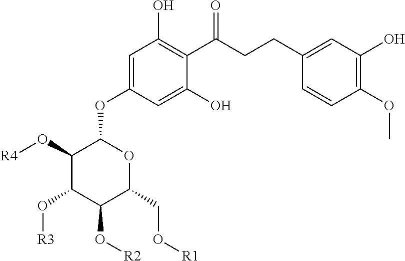

wherein R1, R2, R3 and R4 are independently selected from the group consisting of hydrogen, acetyl residue, propionyl residue, butanoyl residue, pentanoyl residue, hexanoyl residue, heptanoyl residue, octanoyl residue, nonanoyl residue, and decanoyl residue,
wherein at least one of R1, R2, R3 and R4 is not hydrogen,
for providing a sweet taste and/or modulating the sweet taste of one or more sweet tasting substance(s).

It was surprisingly found that the acylation of dihydrochalcone glycoside hesperetin dihydrochalcone-4′-O-β-d-glucoside (HG) increases its sweet taste and its sweetness modulating properties. Thus, the compounds according to formula (I) show an improved sweet taste themselves and improved sweetness modulating properties.

It was surprisingly found that the compounds according to formula (I) provide a similar sweet taste or similar sweetness modulating properties as HG, however already in much lower concentrations.

The term “wherein at least one of R1, R2, R3 and R4 is not hydrogen” is to be understood such that
only one of R1, R2, R3 and R4 is hydrogen,
only two of R1, R2, R3 and R4 are hydrogen, or
only three of R1, R2, R3 and R4 are hydrogen,
   wherein those of R1, R2, R3 and R4, which are not hydrogen, are selected from the group consisting of acetyl residue, propionyl residue, butanoyl residue, pentanoyl residue, hexanoyl residue, heptanoyl residue, octanoyl residue, nonanoyl residue, and decanoyl residue.

Preferably, the use for providing a sweet taste describes the use as a sweetener.

The terms “modulating the sweet taste”, “sweetness modulation” or “sweetness modulating properties” or the like preferably refer to one or more effects selected from increasing the sweet taste, increasing the sweetness onset, increasing the mouthfeel, and altering (such as prolonging or reducing) the lingering sweetness.

Depending on the particular application of the compounds or mixture of compounds as described herein, a prolonged or a reduced lingering sweetness may be desired. Thus, for example, it may be desired to provide a particularly long lasting sweet taste. It was surprisingly found that the compounds or mixture of compounds as described herein are able to alter the lingering sweetness.

The term “modulating the sweet taste of one or more sweet tasting substance(s)” preferably refers to the sweet taste of the one or more sweet tasting substance(s) in a composition comprising the one or more sweet tasting substance(s) and/or the sweet taste of a composition comprising the one or more sweet tasting substance(s).

Preferably, the, one, two, three or more or all compounds of formula (I) is/are selected from

| Compound | R1 | R2 | R3 | R4 |
|---|---|---|---|---|
| 2 | H | H | H | acetyl |
| 3 | H | H | acetyl | H |
| 4 | H | H | acetyl | acetyl |
| 5 | H | acetyl | H | H |
| 6 | H | acetyl | H | acetyl |
| 7 | H | acetyl | acetyl | H |
| 8 | H | acetyl | acetyl | acetyl |
| 9 | acetyl | H | H | H |
| 10 | acetyl | H | H | acetyl |
| 11 | acetyl | H | acetyl | H |
| 12 | acetyl | H | acetyl | acetyl |
| 13 | acetyl | acetyl | H | H |
| 14 | acetyl | acetyl | H | acetyl |
| 15 | acetyl | acetyl | acetyl | H |
| 16 | acetyl | acetyl | acetyl | acetyl |
| 18 | H | H | H | propionyl |
| 19 | H | H | propionyl | H |
| 20 | H | H | propionyl | propionyl |
| 21 | H | propionyl | H | H |
| 22 | H | propionyl | H | propionyl |
| 23 | H | propionyl | propionyl | H |
| 24 | H | propionyl | propionyl | propionyl |
| 25 | propionyl | H | H | H |
| 26 | propionyl | H | H | propionyl |
| 27 | propionyl | H | propionyl | H |
| 28 | propionyl | H | propionyl | propionyl |
| 29 | propionyl | propionyl | H | H |
| 30 | propionyl | propionyl | H | propionyl |
| 31 | propionyl | propionyl | propionyl | H |
| 32 | propionyl | propionyl | propionyl | propionyl |
| 34 | H | H | H | butanoyl |
| 35 | H | H | butanoyl | H |
| 36 | H | H | butanoyl | butanoyl |
| 37 | H | butanoyl | H | H |
| 38 | H | butanoyl | H | butanoyl |
| 39 | H | butanoyl | butanoyl | H |
| 40 | H | butanoyl | butanoyl | butanoyl |
| 41 | butanoyl | H | H | H |
| 42 | butanoyl | H | H | butanoyl |
| 43 | butanoyl | H | butanoyl | H |
| 44 | butanoyl | H | butanoyl | butanoyl |
| 45 | butanoyl | butanoyl | H | H |
| 46 | butanoyl | butanoyl | H | butanoyl |
| 47 | butanoyl | butanoyl | butanoyl | H |
| 48 | butanoyl | butanoyl | butanoyl | butanoyl |
| 50 | H | H | H | pentanoyl |
| 51 | H | H | pentanoyl | H |
| 52 | H | H | pentanoyl | pentanoyl |
| 53 | H | pentanoyl | H | H |
| 54 | H | pentanoyl | H | pentanoyl |
| 55 | H | pentanoyl | pentanoyl | H |
| 56 | H | pentanoyl | pentanoyl | pentanoyl |
| 57 | pentanoyl | H | H | H |
| 58 | pentanoyl | H | H | pentanoyl |
| 59 | pentanoyl | H | pentanoyl | H |
| 60 | pentanoyl | H | pentanoyl | pentanoyl |
| 61 | pentanoyl | pentanoyl | H | H |
| 62 | pentanoyl | pentanoyl | H | pentanoyl |
| 63 | pentanoyl | pentanoyl | pentanoyl | H |
| 64 | pentanoyl | pentanoyl | pentanoyl | pentanoyl |
| 66 | H | H | H | hexanoyl |
| 67 | H | H | hexanoyl | H |
| 68 | H | H | hexanoyl | hexanoyl |
| 69 | H | hexanoyl | H | H |
| 70 | H | hexanoyl | H | hexanoyl |
| 71 | H | hexanoyl | hexanoyl | H |
| 72 | H | hexanoyl | hexanoyl | hexanoyl |
| 73 | hexanoyl | H | H | H |
| 74 | hexanoyl | H | H | hexanoyl |
| 75 | hexanoyl | H | hexanoyl | H |
| 76 | hexanoyl | H | hexanoyl | hexanoyl |
| 77 | hexanoyl | hexanoyl | H | H |
| 78 | hexanoyl | hexanoyl | H | hexanoyl |
| 79 | hexanoyl | hexanoyl | hexanoyl | H |
| 80 | hexanoyl | hexanoyl | hexanoyl | hexanoyl |
| 82 | H | H | H | heptanoyl |
| 83 | H | H | heptanoyl | H |
| 84 | H | H | heptanoyl | heptanoyl |
| 85 | H | heptanoyl | H | H |
| 86 | H | heptanoyl | H | heptanoyl |
| 87 | H | heptanoyl | heptanoyl | H |
| 88 | H | heptanoyl | heptanoyl | heptanoyl |
| 89 | heptanoyl | H | H | H |
| 90 | heptanoyl | H | H | heptanoyl |
| 91 | heptanoyl | H | heptanoyl | H |
| 92 | heptanoyl | H | heptanoyl | heptanoyl |
| 93 | heptanoyl | heptanoyl | H | H |
| 94 | heptanoyl | heptanoyl | H | heptanoyl |
| 95 | heptanoyl | heptanoyl | heptanoyl | H |
| 96 | heptanoyl | heptanoyl | heptanoyl | heptanoyl |
| 98 | H | H | H | octanoyl |
| 99 | H | H | octanoyl | H |
| 100 | H | H | octanoyl | octanoyl |
| 101 | H | octanoyl | H | H |
| 102 | H | octanoyl | H | octanoyl |
| 103 | H | octanoyl | octanoyl | H |
| 104 | H | octanoyl | octanoyl | octanoyl |
| 105 | octanoyl | H | H | H |
| 106 | octanoyl | H | H | octanoyl |
| 107 | octanoyl | H | octanoyl | H |
| 108 | octanoyl | H | octanoyl | octanoyl |
| 109 | octanoyl | octanoyl | H | H |
| 110 | octanoyl | octanoyl | H | octanoyl |
| 111 | octanoyl | octanoyl | octanoyl | H |
| 112 | octanoyl | octanoyl | octanoyl | octanoyl |
| 114 | H | H | H | nonanoyl |
| 115 | H | H | nonanoyl | H |
| 116 | H | H | nonanoyl | nonanoyl |
| 117 | H | nonanoyl | H | H |
| 118 | H | nonanoyl | H | nonanoyl |
| 119 | H | nonanoyl | nonanoyl | H |
| 120 | H | nonanoyl | nonanoyl | nonanoyl |
| 121 | nonanoyl | H | H | H |
| 122 | nonanoyl | H | H | nonanoyl |
| 123 | nonanoyl | H | nonanoyl | H |
| 124 | nonanoyl | H | nonanoyl | nonanoyl |
| 125 | nonanoyl | nonanoyl | H | H |
| 126 | nonanoyl | nonanoyl | H | nonanoyl |
| 127 | nonanoyl | nonanoyl | nonanoyl | H |
| 128 | nonanoyl | nonanoyl | nonanoyl | nonanoyl |
| 130 | H | H | H | decanoyl |
| 131 | H | H | decanoyl | H |
| 132 | H | H | decanoyl | decanoyl |
| 133 | H | decanoyl | H | H |
| 134 | H | decanoyl | H | decanoyl |
| 135 | H | decanoyl | decanoyl | H |
| 136 | H | decanoyl | decanoyl | decanoyl |
| 137 | decanoyl | H | H | H |
| 138 | decanoyl | H | H | decanoyl |
| 139 | decanoyl | H | decanoyl | H |
| 140 | decanoyl | H | decanoyl | decanoyl |
| 141 | decanoyl | decanoyl | H | H |
| 142 | decanoyl | decanoyl | H | decanoyl |
| 143 | decanoyl | decanoyl | decanoyl | H |
| 144 | decanoyl | decanoyl | decanoyl | decanoyl |

Particularly preferably, R2, R3, and R4 are each hydrogen.

Further preferably, the, one, two, three or more or all compounds of formula (I) is/are selected from

| Compound | | |
|---|---|---|
| number | Compound | Structure |
| 41 | 6″-O-Butanoyl-HG | 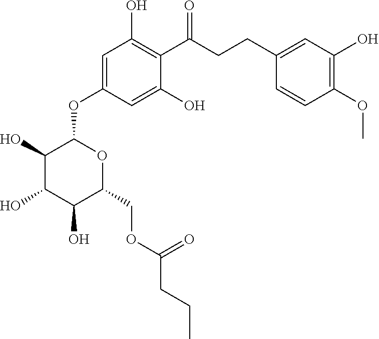 [SMILES: CCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 57 | 6″-O-Pentanoyl-HG | 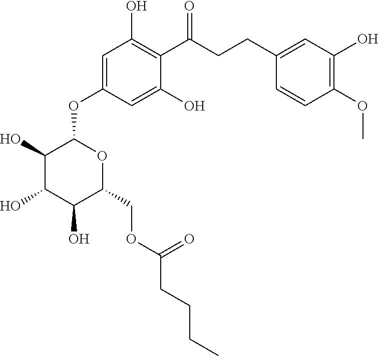 [SMILES: CCCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 73 | 6″-O-Hexanoyl-HG | 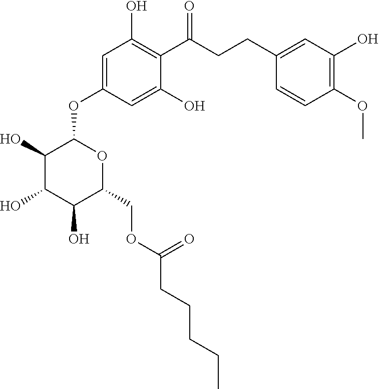 [SMILES: CCCCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 89 | 6″-O-Heptanoyl-HG | 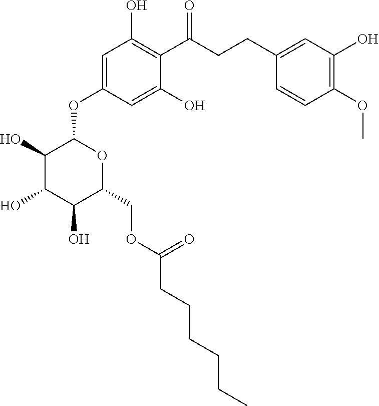 [SMILES: CCCCCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 25 | 6″-O-Propanoyl-HG | 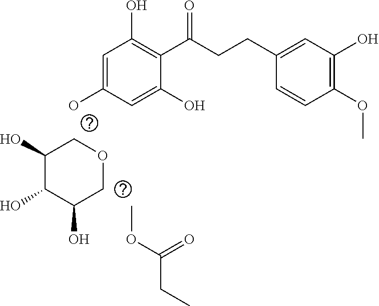 |
| 9 | 6″-O-Acetyl-HG | 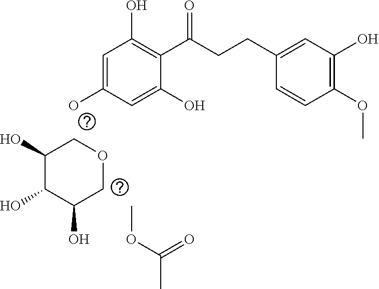 |
| 105 | 6″-O-Octanoyl-HG | 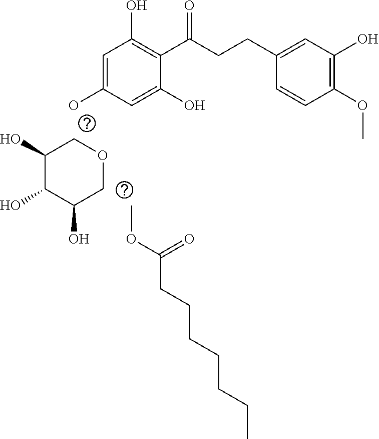 |
| 121 | 6″-O-Nonanoyl-HG | 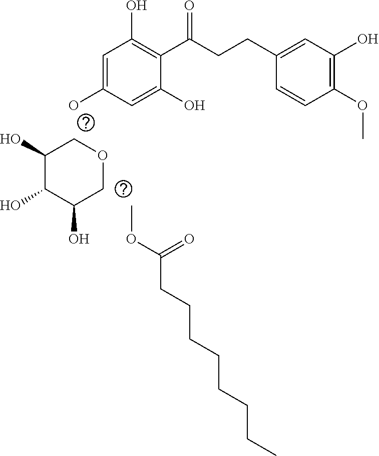 |
| 137 | 6″-O-Decanoyl-HG | 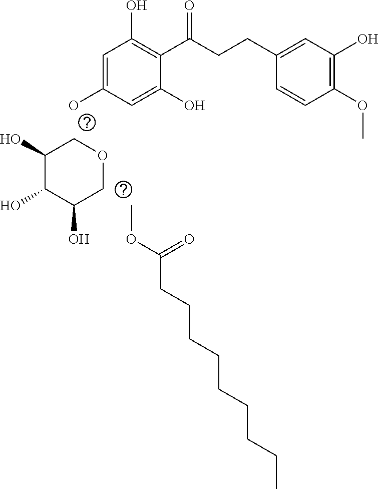 [symbol] |

[symbol]

indicates data missing or illegible when filed

In case a compound is described by a chemical name and a chemical structure and deviations between the name and the structure should exist, the compound shall be described by the provided chemical structure.

The term “sweet tasting substance” refers to any substance, which has a sweet taste, in particular sweeteners, but also to substances, which do not provide a strong enough sweet taste on their own to qualify as sweeteners.

Surprisingly, it was found that the compounds according to formula (I) are able to advantageously modulate the sweet taste of one or more sweet tasting substance(s).

It is further preferred that the, one, two, three or more or all sweet tasting substance(s) is/are selected from the group consisting of
natural sweet tasting substance(s), preferably naturally occurring sweet tasting substances, including plant extracts, such as sweet tasting carbohydrates (such as sucrose, D-(+)-trehalose, lactose, maltose, D-(+)-melizitose, melibiose, D-(+)-raffinose, palatinose, lactulose, D-fructose, D-(+)-allulose, D-glucose, D-galactose, D-(+)-allose, D-(+)-altrose, D-(−)-gulose, D-(+)-mannose, D-(−)-idose, D-(+)-talose, L-(+)-rhamnose, L-(−)-fucose, D-sorbose, D-mannose, D-(−)-tagatose, D-arabinose, L-arabinose, D-(+)-xylose, D-lyxose, D-ribose, D-ribulose, D-(−)-erythrose, D-(+)-threose, D-glyceraldehyde, maltodextrin), sugar alcohols (such as erythritol, threitol, arabitol, ribitol, xylitol, sorbitol, mannitol, maltitol, isomaltit, dulcitol, lactitol), proteins (such as miraculin, pentaidin, monellin, thaumatin, curculin, brazzein, mabinlin), D-amino acids (such as D-phenylalanine, D-tryptophan) or extracts or fractions obtained from natural sources containing these amino acids and/or proteins and the physiologically acceptable salts of these amino acids and/or proteins, particularly the sodium, potassium, calcium or ammonium salts thereof, steviolgylcoside, stevioside, mono-, di-, tri- or tetra-alpha-glycosylated steviosides or rebaudiosides, steviolbiosid, rebaudiosides, rebaudioside A, rebaudioside B, rebaudioside C, rebaudioside D, rebaudioside E, rebaudioside F, rebaudioside G, rebaudioside H, rebaudioside M, rebaudioside N, rebaudioside X, dulcoside, rubusoside, mono-, di-, tri- or tetra-alpha-glycosylated rubusosides, suavioside A, suavioside B, suavioside G, suavioside H, suavioside I, suavioside J, baiyunoside 1, baiyunoside 2, phlomisoside 1, phlomisoside 2, phlomisoside 3, phlomisoside 4, abrusoside A, abrusoside B, abrusoside C, abrusoside D, cyclocaryoside A, cyclocaryoside I, oslandin, polypodoside A, strogin 1, strogin, 2, strogin 4, selligueanin A, dihydroquercetin-3-acetate, perillartin, telosmosid A15, periandrin I-V, pterocaryoside, cyclocaryoside, mukurozioside, trans-anethol, bryoside, bryonoside, bryonodulcoside, carnosifloside, scandenoside, gypenoside, hematoxylin, cyanin, chlorogenic acid, albiziasaponin, telosmoside, gaudichaudiosid, balansin A, balansin B, mogrosides, such as mogroside V, hernandulcine, monatin, glycyrrhetinic acid and its derivatives, particularly glycyrrhizin, preferably glycyrrhizin ammonium salt, extracts or enriched fractions of such extracts such as extracts of Thaumatococcus or Stevia ssp., particularly Stevia rebaudiana, stevia leaf extract, swingle extracts, particularly Momordica or Siratia grosvenorii or LuoHan-Guo, extracts of Glycerrhyzia ssp., particularly Glycerrhyzia glabra or Glycerrhyzia uralensis, extracts of Rubus ssp., particularly Rubus suavissimus or Rubus chingii comprising rubusoside, extracts of Mycetia balansae, preferably comprising balansin A and/or balansin B,
synthetic sweet tasting substances, preferably selected from the group consisting of magap, sodium cyclamate or other physiologically acceptable salts of cyclamic acid, acesulfam K, saccharin, saccharin sodium salt, aspartam, superaspartam, neotam, alitam, advantam, perillartin, sucralose, lugduname, carrelame, sucrononate and sucrooctate.

Particularly preferably, the, one, two, three or more or all sweet tasting substance(s) is/are selected from the group consisting of sucrose, fructose, glucose, steviosides, rebaudiosides, rebaudioside A, rebaudioside M, mono-, di-, tri- or tetra-alpha-glycosylated steviosides or rebaudiosides, rubusoside, mono-, di-, tri- or tetra-alpha-glycosylated rubusosides.

The term “a mixture of two or more compounds of formula (I)” refers to a mixture which comprises or consists of the two or more compounds of formula (I).

Preferably, the compound or mixture of two or more compounds of formula (I) is used, preferably used for modulating the sweet taste of one or more sweet tasting substance(s), in an amount, in which it is perceived as sweet or less sweet than a 5%, preferably a 2.5%, preferably a 1.5% sucrose solution in water.

The compounds of formula (I) can be produced by chemical and enzymatic acylation. HG, hesperetin dihydrochalcone, hesperidin dihydrochalcone or mixtures thereof can be used as starting material. The acylation may be synthetic or enzymatic, as described in the examples below.

The present invention further relates to a method for producing a compound of formula (I), comprising the steps
i) providing one or two or all compounds selected from the group consisting of hesperetin dihydrochalcone, hesperidin dihydrochalcone, and hesperetin dihydrochalcone-4′-O-β-d-glucoside (HG),
ii) providing one or more acyl donor(s),
iii) mixing the compound(s) provided in step i) and the acyl donor(s) provided in step ii) to subject the compound(s) provided in step i) to a chemical or enzymatic acylation.

HG, hesperetin dihydrochalcone, hesperidin dihydrochalcone or mixtures thereof can be provided as plant extracts, such as extracts of Balanophora harlandii.

Thus, it is preferred that in step i) of the method according to the invention, a plant extract comprising one or two or all compounds selected from the group consisting of hesperetin dihydrochalcone, hesperidin dihydrochalcone, and hesperetin dihydrochalcone-4′-O-β-d-glucoside (HG) is provided, preferably wherein the plant extract is an extract of Balanophora harlandii.

As an example for providing such an extract, plant material, in particular leafs of e.g. Balanophora harlandii can be subjected to extraction with a solvent at a temperature from 0° C. to the boiling point of the respective solvent. For the extraction, the ratio of plant material to solvent may be between 1:10 to 1:30, preferably 1:5 to 1:25. Preferably, the solvent is selected from the group consisting of water, subcritical or supercritical water, methanol, ethanol and mixtures thereof. The solvent may be removed and the extract purified by solid-phase adsorption. The adsorbent may be e.g. polystyrene or a mixture of polystyrene and other components. Preferably, water and ethanol are used as solvent with a weight ratio of water:ethanol of at least 1:3, the extraction temperature being between 3° and 50° C., preferably 35 to 45° C. Alternatively, extracts from e.g. Balanophora harlandii plant material are commercially available.

Preferably, the acylation in step iii) of the method according to the invention is a chemical acylation. Such an acylation may be performed according to the following scheme.

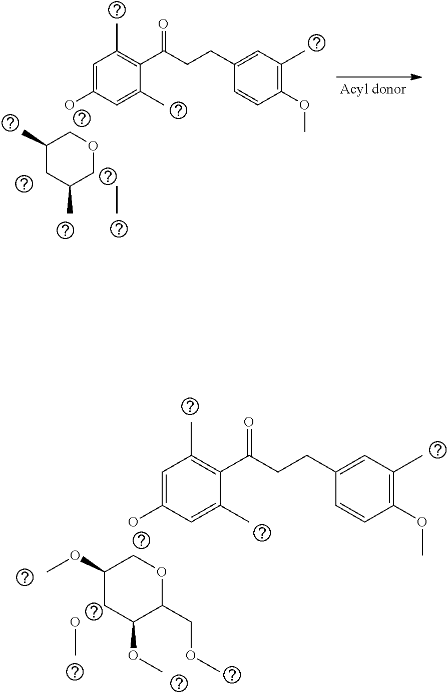

Preferably, the acylation in step iii) of the method according to the invention is an enzymatic acylation In this case, the provided compound(s) selected from the group consisting of hesperetin dihydrochalcone, hesperidin dihydrochalcone, and hesperetin dihydrochalcone-4′-O-β-d-glucoside (HG) is/are incubated with an acyl donor and an enzyme, preferably a lipase.

Thus, preferably, the acylation in step iii) is an enzymatic acylation and wherein the enzyme is an esterase, preferably a lipase, preferably wherein the lipase is a lipase obtained from a microorganism selected from the group consisting of Candida antarctica A, Candida antarctica B, Candida rugosa, Burkholderia cepacia, Rhizopus sp., Rhizomucor miehei, Mucor javanicus, Yarrowia lypolytica, Geotrichum candidum, Aspergillus niger, Aspergillus oryzae, Pseudomonas alcaligenes, Pseudomonas mendocina, Thermomyces lanuginosus and Chromobacterium viscosum.

Preferably, the or one, two, three or more or all acyl donor(s) is/are selected from the group consisting of ethanoyl donors, propanoyl donors, butanoyl donors, pentanoyl donors, hexanoyl donors, heptanoyl donors, octanoyl donors, nonanoyl donors, and decanoyl donors.

Typically, for producing a compound as described herein, the or one acyl donor(s) is/are esters with a corresponding acyl residue. For example, if a compound having a propionyl residue at R1, R2, R3 and/or R4 shall be produced, the or one acyl donor(s) is an ester with a propionyl residue.

Preferably, ethanoyl donors as described herein are selected from the group consisting of triacetin, acetic acid, methyl acetate, ethyl acetate, acetic anhydride and mixtures thereof.

Preferably, propanoyl donors as described herein are selected from the group consisting of tripropionin, propionic acid, methyl propanoate, ethyl propanoate, and mixtures thereof.

Preferably, butanoyl donors as described herein are selected from the group consisting of tributyrin, butanoic acid, methyl butanoate, ethyl butanoate, and mixtures thereof.

Preferably, pentanoyl donors as described herein are selected from the group consisting of tripentanoin, pentanoic acid, methyl pentanoate, ethyl pentanoate, and mixtures thereof.

Preferably, hexanoyl donors as described herein are selected from the group consisting of trihexanoin, hexanoic acid, methyl hexanoate, ethyl hexanoate, and mixtures thereof.

Preferably, heptanoyl donors as described herein are selected from the group consisting of triheptanoin, heptanoic acid, methyl heptanoate, ethyl heptanoate, and mixtures thereof.

Preferably, octanoyl donors as described herein are selected from the group consisting of trioctanoin, octanoic acid, methyl octanoate, ethyl octanoate, and mixtures thereof.

Preferably, nonanoyl donors as described herein are selected from the group consisting of trinonanoin, nonanoic acid, methyl nonanoate, ethyl nonanoate, and mixtures thereof.

Preferably, decanoyl donors as described herein are selected from the group consisting of tricaprin, decanoic acid, methyl decanoate, ethyl decanoate, and mixtures thereof.

Preferably, the or one, two, three or more or all acyl donor(s) is/are selected from the group consisting of triacetin, acetic acid, methyl acetate, ethyl acetate, acetic anhydride, tripropionin, propionic acid, methyl propanoate, ethyl propanoate, tributyrin, butanoic acid, methyl butanoate, ethyl butanoate, tripentanoin, pentanoic acid, methyl pentanoate, ethyl pentanoate, trihexanoin, hexanoic acid, methyl hexanoate, ethyl hexanoate, triheptanoin, heptanoic acid, methyl heptanoate, ethyl heptanoate, trioctanoin, octanoic acid, methyl octanoate, ethyl octanoate, trinonanoin, nonanoic acid, methyl nonanoate, ethyl nonanoate, tricaprin, decanoic acid, methyl decanoate, and ethyl decanoate.

The present invention further relates to a compound according to formula (I)

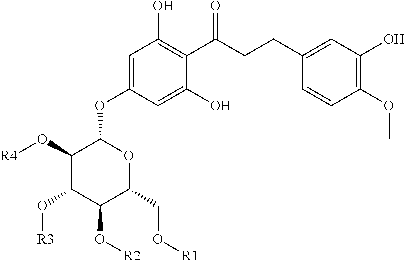

wherein the compound is selected from

| Compound | R1 | R2 | R3 | R4 |
|---|---|---|---|---|
| 2 | H | H | H | acetyl |
| 3 | H | H | acetyl | H |
| 4 | H | H | acetyl | acetyl |
| 5 | H | acetyl | H | H |
| 6 | H | acetyl | H | acetyl |
| 7 | H | acetyl | acetyl | H |
| 8 | H | acetyl | acetyl | acetyl |
| 9 | acetyl | H | H | H |
| 10 | acetyl | H | H | acetyl |
| 11 | acetyl | H | acetyl | H |
| 12 | acetyl | H | acetyl | acetyl |
| 13 | acetyl | acetyl | H | H |
| 14 | acetyl | acetyl | H | acetyl |
| 15 | acetyl | acetyl | acetyl | H |
| 16 | acetyl | acetyl | acetyl | acetyl |
| 18 | H | H | H | propionyl |
| 19 | H | H | propionyl | H |
| 20 | H | H | propionyl | propionyl |
| 21 | H | propionyl | H | H |
| 22 | H | propionyl | H | propionyl |
| 23 | H | propionyl | propionyl | H |
| 24 | H | propionyl | propionyl | propionyl |
| 25 | propionyl | H | H | H |
| 26 | propionyl | H | H | propionyl |
| 27 | propionyl | H | propionyl | H |
| 28 | propionyl | H | propionyl | propionyl |
| 29 | propionyl | propionyl | H | H |
| 30 | propionyl | propionyl | H | propionyl |
| 31 | propionyl | propionyl | propionyl | H |
| 32 | propionyl | propionyl | propionyl | propionyl |
| 34 | H | H | H | butanoyl |
| 35 | H | H | butanoyl | H |
| 36 | H | H | butanoyl | butanoyl |
| 37 | H | butanoyl | H | H |
| 38 | H | butanoyl | H | butanoyl |
| 39 | H | butanoyl | butanoyl | H |
| 40 | H | butanoyl | butanoyl | butanoyl |
| 41 | butanoyl | H | H | H |
| 42 | butanoyl | H | H | butanoyl |
| 43 | butanoyl | H | butanoyl | H |
| 44 | butanoyl | H | butanoyl | butanoyl |
| 45 | butanoyl | butanoyl | H | H |
| 46 | butanoyl | butanoyl | H | butanoyl |
| 47 | butanoyl | butanoyl | butanoyl | H |
| 48 | butanoyl | butanoyl | butanoyl | butanoyl |
| 50 | H | H | H | pentanoyl |
| 51 | H | H | pentanoyl | H |
| 52 | H | H | pentanoyl | pentanoyl |
| 53 | H | pentanoyl | H | H |
| 54 | H | pentanoyl | H | pentanoyl |
| 55 | H | pentanoyl | pentanoyl | H |
| 56 | H | pentanoyl | pentanoyl | pentanoyl |
| 57 | pentanoyl | H | H | H |
| 58 | pentanoyl | H | H | pentanoyl |
| 59 | pentanoyl | H | pentanoyl | H |
| 60 | pentanoyl | H | pentanoyl | pentanoyl |
| 61 | pentanoyl | pentanoyl | H | H |
| 62 | pentanoyl | pentanoyl | H | pentanoyl |
| 63 | pentanoyl | pentanoyl | pentanoyl | H |
| 64 | pentanoyl | pentanoyl | pentanoyl | pentanoyl |
| 66 | H | H | H | hexanoyl |
| 67 | H | H | hexanoyl | H |
| 68 | H | H | hexanoyl | hexanoyl |
| 69 | H | hexanoyl | H | H |
| 70 | H | hexanoyl | H | hexanoyl |
| 71 | H | hexanoyl | hexanoyl | H |
| 72 | H | hexanoyl | hexanoyl | hexanoyl |
| 73 | hexanoyl | H | H | H |
| 74 | hexanoyl | H | H | hexanoyl |
| 75 | hexanoyl | H | hexanoyl | H |
| 76 | hexanoyl | H | hexanoyl | hexanoyl |
| 77 | hexanoyl | hexanoyl | H | H |
| 78 | hexanoyl | hexanoyl | H | hexanoyl |
| 79 | hexanoyl | hexanoyl | hexanoyl | H |
| 80 | hexanoyl | hexanoyl | hexanoyl | hexanoyl |
| 82 | H | H | H | heptanoyl |
| 83 | H | H | heptanoyl | H |
| 84 | H | H | heptanoyl | heptanoyl |
| 85 | H | heptanoyl | H | H |
| 86 | H | heptanoyl | H | heptanoyl |
| 87 | H | heptanoyl | heptanoyl | H |
| 88 | H | heptanoyl | heptanoyl | heptanoyl |
| 89 | heptanoyl | H | H | H |
| 90 | heptanoyl | H | H | heptanoyl |
| 91 | heptanoyl | H | heptanoyl | H |
| 92 | heptanoyl | H | heptanoyl | heptanoyl |
| 93 | heptanoyl | heptanoyl | H | H |
| 94 | heptanoyl | heptanoyl | H | heptanoyl |
| 95 | heptanoyl | heptanoyl | heptanoyl | H |
| 96 | heptanoyl | heptanoyl | heptanoyl | heptanoyl |
| 98 | H | H | H | octanoyl |
| 99 | H | H | octanoyl | H |
| 100 | H | H | octanoyl | octanoyl |
| 101 | H | octanoyl | H | H |
| 102 | H | octanoyl | H | octanoyl |
| 103 | H | octanoyl | octanoyl | H |
| 104 | H | octanoyl | octanoyl | octanoyl |
| 105 | octanoyl | H | H | H |
| 106 | octanoyl | H | H | octanoyl |
| 107 | octanoyl | H | octanoyl | H |
| 108 | octanoyl | H | octanoyl | octanoyl |
| 109 | octanoyl | octanoyl | H | H |
| 110 | octanoyl | octanoyl | H | octanoyl |
| 111 | octanoyl | octanoyl | octanoyl | H |
| 112 | octanoyl | octanoyl | octanoyl | octanoyl |
| 114 | H | H | H | nonanoyl |
| 115 | H | H | nonanoyl | H |
| 116 | H | H | nonanoyl | nonanoyl |
| 117 | H | nonanoyl | H | H |
| 118 | H | nonanoyl | H | nonanoyl |
| 119 | H | nonanoyl | nonanoyl | H |
| 120 | H | nonanoyl | nonanoyl | nonanoyl |
| 121 | nonanoyl | H | H | H |
| 122 | nonanoyl | H | H | nonanoyl |
| 123 | nonanoyl | H | nonanoyl | H |
| 124 | nonanoyl | H | nonanoyl | nonanoyl |
| 125 | nonanoyl | nonanoyl | H | H |
| 126 | nonanoyl | nonanoyl | H | nonanoyl |
| 127 | nonanoyl | nonanoyl | nonanoyl | H |
| 128 | nonanoyl | nonanoyl | nonanoyl | nonanoyl |
| 130 | H | H | H | decanoyl |
| 131 | H | H | decanoyl | H |
| 132 | H | H | decanoyl | decanoyl |
| 133 | H | decanoyl | H | H |
| 134 | H | decanoyl | H | decanoyl |
| 135 | H | decanoyl | decanoyl | H |
| 136 | H | decanoyl | decanoyl | decanoyl |
| 137 | decanoyl | H | H | H |
| 138 | decanoyl | H | H | decanoyl |
| 139 | decanoyl | H | decanoyl | H |
| 140 | decanoyl | H | decanoyl | decanoyl |
| 141 | decanoyl | decanoyl | H | H |
| 142 | decanoyl | decanoyl | H | decanoyl |
| 143 | decanoyl | decanoyl | decanoyl | H |
| 144 | decanoyl | decanoyl | decanoyl | decanoyl |

As described herein, it was surprisingly found that the acylation of dihydrochalcone glycoside hesperetin dihydrochalcone-4′-O-β-d-glucoside (HG) increases its sweet taste and its sweetness modulating properties. Thus, the compounds according to formula (I) show an improved sweet taste themselves and improved sweetness modulating properties.

It was surprisingly found that the compounds according to formula (I) provide a similar sweet taste or similar sweetness modulating properties as HG, however already in much lower concentrations.

Preferably, R2, R3, and R4 of the compound according to the invention are each hydrogen. Thus, R1 is selected from the group consisting of acetyl residue, propionyl residue, butanoyl residue, pentanoyl residue, hexanoyl residue, and heptanoyl residue.

Further preferably, the compound according to the invention is selected from

| Compound | | |
|---|---|---|
| number | Compound | Structure |
| 41 | 6″-O-Butanoyl-HG | 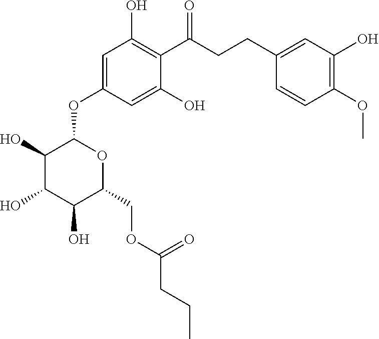 [SMILES: CCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 57 | 6″-O-Pentanoyl-HG | 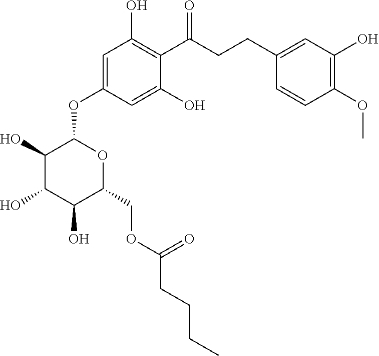 [SMILES: CCCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 73 | 6″-O-Hexanoyl-HG | 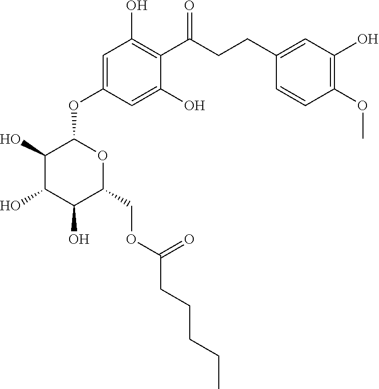 [SMILES: CCCCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 89 | 6″-O-Heptanoyl-HG | 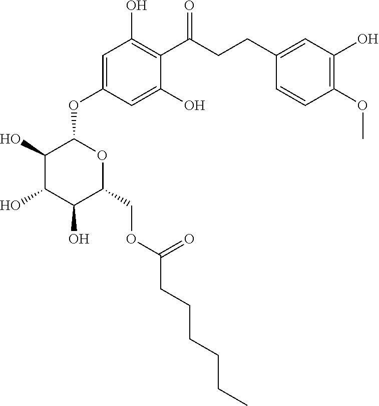 [SMILES: CCCCCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 25 | 6″-O-Propanoyl-HG | 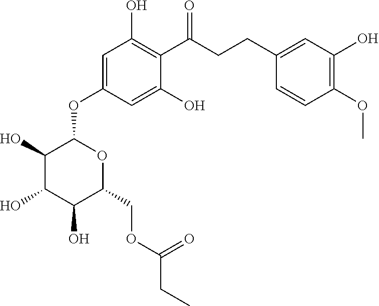 [SMILES: CCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 9 | 6″-O-Acetyl-HG | 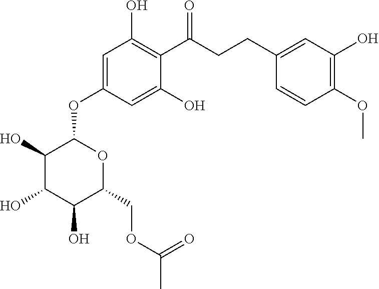 [SMILES: COc1ccc(CCC(=O)c2c(O)cc(O[C@@H]3O[C@H](COC(C)=O)[C@@H](O)[C@H](O)[C@H]3O)cc2O)cc1O] |
| 105 | 6″-O-Octanoyl-HG | 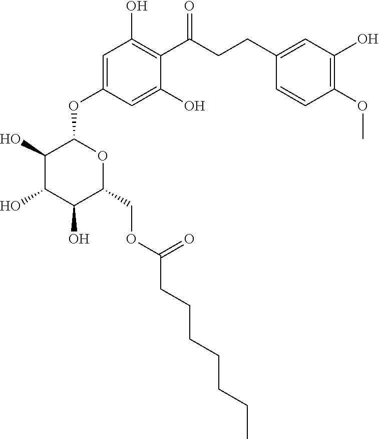 [SMILES: CCCCCCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 121 | 6″-O-Nonanoyl-HG | 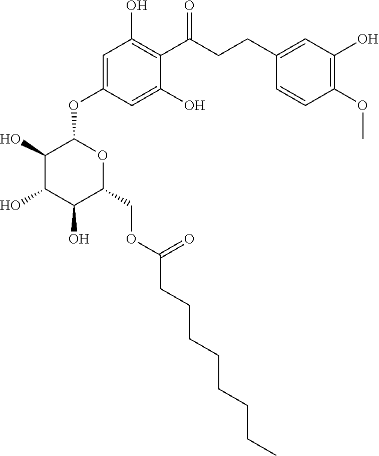 [SMILES: CCCCCCCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 137 | 6″-O-Decanoyl-HG | 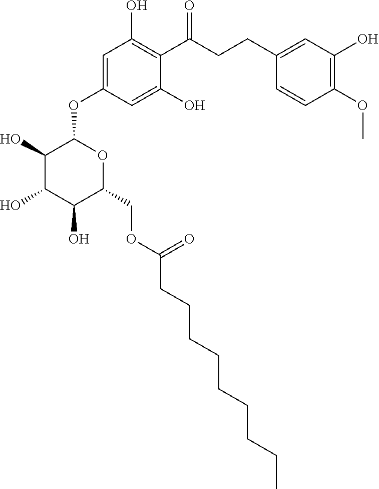 [SMILES: CCCCCCCCCC(=O)OC[C@H]1O[C@@H](Oc2cc(O)c(C(=O)CCc3ccc(OC)c(O)c3)c(O)c2)[C@H](O)[C@@H](O)[C@@H]1O] |

The present invention further relates to a composition comprising a compound according to the invention.

Preferably, the composition further comprises one or more sweet tasting substance(s) selected from the group consisting of
natural sweet tasting substance(s), preferably naturally occurring sweet tasting substances, including plant extracts, such as sweet tasting carbohydrates (such as sucrose, D-(+)-trehalose, lactose, maltose, D-(+)-melizitose, melibiose, D-(+)-raffinose, palatinose, lactulose, D-fructose, D-(+)-allulose, D-glucose, D-galactose, D-(+)-allose, D-(+)-altrose, D-(−)-gulose, D-(+)-mannose, D-(−)-idose, D-(+)-talose, L-(+)-rhamnose, L-(−)-fucose, D-sorbose, D-mannose, D-(−)-tagatose, D-arabinose, L-arabinose, D-(+)-xylose, D-lyxose, D-ribose, D-ribulose, D-(−)-erythrose, D-(+)-threose, D-glyceraldehyde, maltodextrin), sugar alcohols (such as erythritol, threitol, arabitol, ribitol, xylitol, sorbitol, mannitol, maltitol, isomaltit, dulcitol, lactitol), proteins (such as miraculin, pentaidin, monellin, thaumatin, curculin, brazzein, mabinlin), D-amino acids (such as D-phenylalanine, D-tryptophan) or extracts or fractions obtained from natural sources containing these amino acids and/or proteins and the physiologically acceptable salts of these amino acids and/or proteins, particularly the sodium, potassium, calcium or ammonium salts thereof, steviolgylcoside, stevioside, mono-, di-, tri- or tetra-alpha-glycosylated steviosides or rebaudiosides, steviolbiosid, rebaudiosides, rebaudioside A, rebaudioside B, rebaudioside C, rebaudioside D, rebaudioside E, rebaudioside F, rebaudioside G, rebaudioside H, rebaudioside M, rebaudioside N, rebaudioside X, dulcoside, rubusoside, mono-, di-, tri- or tetra-alpha-glycosylated rubusosides, suavioside A, suavioside B, suavioside G, suavioside H, suavioside I, suavioside J, baiyunoside 1, baiyunoside 2, phlomisoside 1, phlomisoside 2, phlomisoside 3, phlomisoside 4, abrusoside A, abrusoside B, abrusoside C, abrusoside D, cyclocaryoside A, cyclocaryoside I, oslandin, polypodoside A, strogin 1, strogin, 2, strogin 4, selligueanin A, dihydroquercetin-3-acetate, perillartin, telosmosid A15, periandrin I-V, pterocaryoside, cyclocaryoside, mukurozioside, trans-anethol, bryoside, bryonoside, bryonodulcoside, carnosifloside, scandenoside, gypenoside, hematoxylin, cyanin, chlorogenic acid, albiziasaponin, telosmoside, gaudichaudiosid, balansin A, balansin B, mogrosides, such as mogroside V, hernandulcine, monatin, glycyrrhetinic acid and its derivatives, particularly glycyrrhizin, preferably glycyrrhizin ammonium salt, extracts or enriched fractions of such extracts such as extracts of Thaumatococcus or Stevia ssp., particularly Stevia rebaudiana, stevia leaf extract, swingle extracts, particularly Momordica or Siratia grosvenorii or LuoHan-Guo, extracts of Glycerrhyzia ssp., particularly Glycerrhyzia glabra or Glycerrhyzia uralensis, extracts of Rubus ssp., particularly Rubus suavissimus or Rubus chingii comprising rubusoside, extracts of Mycetia balansae, preferably comprising balansin A and/or balansin B,
synthetic sweet tasting substances, preferably selected from the group consisting of magap, sodium cyclamate or other physiologically acceptable salts of cyclamic acid, acesulfam K, saccharin, saccharin sodium salt, aspartam, superaspartam, neotam, alitam, advantam, perillartin, sucralose, lugduname, carrelame, sucrononate and sucrooctate.

Particularly preferably, the composition comprises one or more sweet tasting substance(s) selected from the group consisting of sucrose, fructose, glucose, steviosides, rebaudiosides, rebaudioside A, rebaudioside M, mono-, di-, tri- or tetra-alpha-glycosylated steviosides or rebaudiosides, rubusoside, mono-, di-, tri- or tetra-alpha-glycosylated rubusosides.

Particularly preferably, the composition comprises one or more sweet taste modulating substance(s) selected from the group consisting of hesperetin, hesperetin dihydrochalcone, naringenin, phloretin, eriodictyol, homoeriodictyol, phyllodulcin, neohesperidindihydrochalkon, naringindihydrochalkon, phloretin, extracts of Hydrangea macrophylla ssp. serrata, in particular var. Oamacha, Amacha or Amagi amacha comprising active amounts of phyllodulcin.

It was further surprisingly found that combining two or more of the compounds of formula (I) provides a synergistic effect.

It is thus preferred that the composition according to the invention comprises two or more compounds of formula (I).

It is thus preferred that the composition according to the invention comprises two or more compounds of formula (I), wherein at least two of R1, R2, R3, and R4 are an acetyl residue, preferably wherein R1 and at least one of R2, R3, and R4 are an acetyl residue.

It is thus preferred that the composition according to the invention comprises two or more compounds of formula (I), wherein at least two of R1, R2, R3, and R4 are a propionyl residue, preferably wherein R1 and at least one of R2, R3, and R4 are a propionyl residue.

It is thus preferred that the composition according to the invention comprises two or more compounds of formula (I), wherein at least two of R1, R2, R3, and R4 are a butanoyl residue, preferably wherein R1 and at least one of R2, R3, and R4 are a butanoyl residue.

It is thus preferred that the composition according to the invention comprises two or more compounds of formula (I), wherein at least two of R1, R2, R3, and R4 are a pentanoyl residue, preferably wherein R1 and at least one of R2, R3, and R4 are a pentanoyl residue.

It is thus preferred that the composition according to the invention comprises two or more compounds of formula (I), wherein at least two of R1, R2, R3, and R4 are a hexanoyl residue, preferably wherein R1 and at least one of R2, R3, and R4 are a hexanoyl residue.

It is thus preferred that the composition according to the invention comprises two or more compounds of formula (I), wherein at least two of R1, R2, R3, and R4 are a heptanoyl residue, preferably wherein R1 and at least one of R2, R3, and R4 are a heptanoyl residue.

It is thus preferred that the composition according to the invention comprises two or more compounds of formula (I), wherein at least two of R1, R2, R3, and R4 are an octanoyl residue, preferably wherein R1 and at least one of R2, R3, and R4 are an octanoyl residue.

It is thus preferred that the composition according to the invention comprises two or more compounds of formula (I), wherein at least two of R1, R2, R3, and R4 are a nonanoyl residue, preferably wherein R1 and at least one of R2, R3, and R4 are a nonanoyl residue.

It is thus preferred that the composition according to the invention comprises two or more compounds of formula (I), wherein at least two of R1, R2, R3, and R4 are a decanoyl residue, preferably wherein R1 and at least one of R2, R3, and R4 are a decanoyl residue.

It is further preferred that the composition according to the invention comprises two or more compounds of formula (I), wherein the composition comprises compound 9 and compound 25.

It is further preferred that the composition according to the invention comprises two or more compounds of formula (I), wherein the composition comprises compound 9 and compound 41.

It is further preferred that the composition according to the invention comprises two or more compounds of formula (I), wherein the composition comprises compound 9 and compound 57.

It is further preferred that the composition according to the invention comprises two or more compounds of formula (I), wherein the composition comprises compound 9 and compound 73.

It is further preferred that the composition according to the invention comprises two or more compounds of formula (I), wherein the composition comprises compound 9 and compound 89.

It is further preferred that the composition according to the invention comprises two or more compounds of formula (I), wherein the composition comprises compound 9 and compound 105.

It is further preferred that the composition according to the invention comprises two or more compounds of formula (I), wherein the composition comprises compound 9 and compound 121.

It is further preferred that the composition according to the invention comprises two or more compounds of formula (I), wherein the composition comprises compound 9 and compound 137.

It is further preferred that the composition according to the invention comprises two or more compounds of formula (I), wherein the composition comprises compound 25 and compound 41.

It is further preferred that the composition according to the invention comprises two or more compounds of formula (I), wherein the composition comprises compound 25 and compound 57.

It is further preferred that the composition according to the invention comprises two or more compounds of formula (I), wherein the composition comprises compound 25 and compound 73.

It is further preferred that the composition according to the invention comprises two or more compounds of formula (I), wherein the composition comprises compound 25 and compound 89.

It is further preferred that the composition according to the invention comprises two or more compounds of formula (I), wherein the composition comprises compound 25 and compound 105.

It is further preferred that the composition according to the invention comprises two or more compounds of formula (I), wherein the composition comprises compound 25 and compound 121.

It is further preferred that the composition according to the invention comprises two or more compounds of formula (I), wherein the composition comprises compound 25 and compound 137.

It is further preferred that the composition according to the invention comprises two or more compounds of formula (I), wherein the composition comprises compound 41 and compound 57.

It is further preferred that the composition according to the invention comprises two or more compounds of formula (I), wherein the composition comprises compound 41 and compound 73.

It is further preferred that the composition according to the invention comprises two or more compounds of formula (I), wherein the composition comprises compound 41 and compound 89.

It is further preferred that the composition according to the invention comprises two or more compounds of formula (I), wherein the composition comprises compound 41 and compound 105.

It is further preferred that the composition according to the invention comprises two or more compounds of formula (I), wherein the composition comprises compound 41 and compound 121.

It is further preferred that the composition according to the invention comprises two or more compounds of formula (I), wherein the composition comprises compound 41 and compound 137.

It is further preferred that the composition according to the invention comprises two or more compounds of formula (I), wherein the composition comprises compound 57 and compound 73.

It is further preferred that the composition according to the invention comprises two or more compounds of formula (I), wherein the composition comprises compound 57 and compound 89.

It is further preferred that the composition according to the invention comprises two or more compounds of formula (I), wherein the composition comprises compound 57 and compound 105.

It is further preferred that the composition according to the invention comprises two or more compounds of formula (I), wherein the composition comprises compound 57 and compound 121.

It is further preferred that the composition according to the invention comprises two or more compounds of formula (I), wherein the composition comprises compound 57 and compound 137.

It is further preferred that the composition according to the invention comprises two or more compounds of formula (I), wherein the composition comprises compound 73 and compound 89.

It is further preferred that the composition according to the invention comprises two or more compounds of formula (I), wherein the composition comprises compound 73 and compound 105.

It is further preferred that the composition according to the invention comprises two or more compounds of formula (I), wherein the composition comprises compound 73 and compound 121.

It is further preferred that the composition according to the invention comprises two or more compounds of formula (I), wherein the composition comprises compound 73 and compound 137.

It is further preferred that the composition according to the invention comprises two or more compounds of formula (I), wherein the composition comprises compound 89 and compound 105.

It is further preferred that the composition according to the invention comprises two or more compounds of formula (I), wherein the composition comprises compound 89 and compound 121.

It is further preferred that the composition according to the invention comprises two or more compounds of formula (I), wherein the composition comprises compound 89 and compound 137.

It is further preferred that the composition according to the invention comprises two or more compounds of formula (I), wherein the composition comprises compound 105 and compound 121.

It is further preferred that the composition according to the invention comprises two or more compounds of formula (I), wherein the composition comprises compound 105 and compound 137.

It is further preferred that the composition according to the invention comprises two or more compounds of formula (I), wherein the composition comprises compound 121 and compound 137.

Preferably, the amount of the compound(s) according to formula (I) in the composition is in a range of from 0.0001 to 75 wt.-%, preferably in a range of from 0.0002 to 50 wt.-%, preferably in a range of from 0.0003 to 25 wt.-%, preferably in a range of from 0.0005 to 10 wt. %, preferably in a range of from 0.00075 to 5 wt.-%, preferably in a range of from 0.001 to 1 wt.-%, based on the total weight of the composition.

Preferably, the term “the amount of the compound(s) according to formula (I) in the composition” refers to the summed amount of all compound(s) according to formula (I), present in the composition.

The invention further relates to a product comprising the composition according to the invention.

Preferably, the amount of the compound(s) according to formula (I) in the product is in a range of from 0.0001 to 1 wt.-%, preferably in a range of from 0.0002 to 0.1 wt.-%, preferably in a range of from 0.0003 to 0.01 wt.-%, preferably in a range of from 0.0005 to 0.005 wt.-%, based on the total weight of the product.

Preferably, the amount of the compound(s) according to formula (I) in the product is at most 50 ppm, preferably at most 25 ppm, particularly preferably at most 20 ppm, especially preferably at most 10 ppm, further preferably at most 7.5 ppm, even further preferably at most 5 ppm with respect to the total product.

Preferably, the amount of the compound(s) according to formula (I) in the product at least 3 ppm, preferably at least 5 ppm, particularly preferably at least 10 ppm, especially preferably at least 15 ppm, further preferably at least 20 ppm, more preferably at least 25 ppm, even further preferably at least 50 ppm, with respect to the total product.

Preferably, the term “the amount of the compound(s) according to formula (I) in the product” refers to the summed amount of all compound(s) according to formula (I), present in the product.

The product according to the invention can be selected from the group consisting of pharmaceutical products for oral application, oral care products, liquid and solid products for nutrition or pleasure and semi-finished products.

Preferably, a semi-finished product refers to a substance, product or good that has not completed the manufacturing or production process and is not ready for sale to or use or consumption by consumers and/or clients in the food service or catering industry. Thus, a semi-finished product preferably refers to a product, good or article that is intended for further industrial processing and/or use in industrial processing.

Preferably, a semi-finished product is to be understood as a product, which is unsuitable for use as a ready-to-eat foodstuff. Only by mixing with at least one other ingredient (e.g. to reduce the concentration of flavours) and optionally further processing steps (e.g., heating, freezing) is the semi-finished product converted into a ready-to-eat food.

Preferably, semi-finished products are selected from substances, products or goods intended for use in food products, such as aroma compositions, extracts, mash, powder, oil or paste made from vegetables or fruit, spices, toppings, seasoning mixes, granulated or pulverized foodstuffs.

Preferably, the amount of the compound(s) according to formula (I) in the semi-finished product is in a range of from 0.0001 to 75 wt.-%, preferably in a range of from 0.0002 to 50 wt.-%, preferably in a range of from 0.0003 to 25 wt.-%, preferably in a range of from 0.0005 to 10 wt.-%, preferably in a range of from 0.00075 to 5 wt.-%, preferably in a range of from 0.001 to 1 wt.-%, based on the total weight of the semi-finished product.

Preferably, the products for nutrition or pleasure may be selected from the group consisting of (reduced-calorie) baked goods (e.g. bread, dry biscuits, cakes, other baked articles), confectionary (e.g. muesli bar products, chocolates, chocolate bars, other products in bar form, fruit gums, dragees, hard and soft caramels, chewing gum), non-alcoholic drinks (e.g. cocoa, coffee, green tea, black tea, (green, black) tea drinks enriched with (green, black) tea extracts, rooibos tea, other herbal teas, fruit-containing soft drinks, isotonic drinks, refreshing drinks, nectars, fruit and vegetable juices, fruit or vegetable juice preparations), instant drinks (e.g. instant cocoa drinks, instant tea drinks, instant coffee drinks), meat products (e.g. ham, fresh sausage or raw sausage preparations, spiced or marinated fresh or salt meat products), eggs or egg products (dried egg, egg white, egg yolk), cereal products (e.g. breakfast cereals, muesli bars, precooked ready-to-eat rice products), dairy products (e.g. full-fat or reduced-fat or fat-free milk drinks, rice pudding, yoghurt, kefir, cream cheese, soft cheese, hard cheese, dried milk powder, whey, butter, buttermilk, ice-cream, partially or completely hydrolysed milk-protein-containing products), products made from soy protein or other soybean fractions (e.g. soy milk and products produced therefrom, drinks containing isolated or enzymatically treated soy protein, drinks containing soy flour, preparations containing soy lecithin, fermented products such as tofu or tempeh or products produced therefrom and mixtures with fruit preparations and optionally flavours), dairy-like preparations (milk-type, yoghurt-type, dessert-type, ice cream) from protein rich plant materials (e.g. from seed materials of oat, almond, pea, lupine, lentils, faba beans, chickpea, rice, canola), plant protein-enriched non-dairy drinks, fruit preparations (e.g. jams, sorbets, fruit sauces, fruit fillings), vegetable preparations (e.g. ketchup, sauces, dried vegetables, frozen vegetables, precooked vegetables, boiled-down vegetables), snacks (e.g. baked or fried potato crisps or potato dough products, maize- or groundnut-based extrudates), fat- and oil-based products or emulsions thereof (e.g. mayonnaise, remoulade, dressings, in each case full-fat or reduced-fat), other ready-made dishes and soups (e.g. dried soups, instant soups, precooked soups), spices, spice mixtures and in particular seasonings which are used, for example, in the snacks field, sweetener preparations, tablets or sachets, other preparations for sweetening or whitening drinks.

The product can be a dietary supplement or pharmaceutical product in the form of capsules, tablets (uncoated and coated tablets, e.g. gastro-resistant coatings), sugar-coated pills, granulates, pellets, solid mixtures, dispersions in liquid phases, as emulsions, as powders, as solutions, as pastes or as other formulations that can be swallowed or chewed.

Oral care products are formulations commonly used by those skilled in the art for cleaning and caring for the oral cavity and pharynx and for freshening the breath. Known and common oral care products are in the form of creams, gels, pastes, foams, emulsions, suspensions, aerosols, sprays as well as capsules, granules, pastilles, tablets, sweets or chewing gums, without this list of dosage forms being limiting with regard to the possible applications. Such formulations are used to clean and care for the tooth structure and oral cavity and to freshen the breath. In particular, oral care products according to the invention are preferably selected from the group consisting of: toothpastes, tooth gels, mouthwashes, mouth rinses, liquids for gargling, oral or pharyngeal sprays (pump or aerosol spray), lozenges, lozenges, candies, chewing gums, chewy candies and dental care chewing gums.

Preferably, the unit “ppm” refers to the weight and describes for example a unit corresponding to mg/kg.

Furthermore, the present invention relates to a method for modulating the sweet taste of
one or more sweet tasting substance(s), comprising the steps
a) providing one or more compound according to the invention,
b) providing one or more sweet tasting substance(s), and
c) mixing the compound(s) provided in step a) with the one or more sweet tasting substance(s) provided in step b),
   preferably wherein the, one, two, three or more or all sweet tasting substance(s) is/are selected from the group consisting of
      natural sweet tasting substance(s), preferably naturally occurring sweet tasting substances, including plant extracts, such as sweet tasting carbohydrates (such as sucrose, D-(+)-trehalose, lactose, maltose, D-(+)-melizitose, melibiose, D-(+)-raffinose, palatinose, lactulose, D-fructose, D-(+)-allulose, D-glucose, D-galactose, D-(+)-allose, D-(+)-altrose, D-(−)-gulose, D-(+)-mannose, D-(−)-idose, D-(+)-talose, L-(+)-rhamnose, L-(−)-fucose, D-sorbose, D-mannose, D-(−)-tagatose, D-arabinose, L-arabinose, D-(+)-xylose, D-lyxose, D-ribose, D-ribulose, D-(−)-erythrose, D-(+)-threose, D-glyceraldehyde, maltodextrin), sugar alcohols (such as erythritol, threitol, arabitol, ribitol, xylitol, sorbitol, mannitol, maltitol, isomaltit, dulcitol, lactitol), proteins (such as miraculin, pentaidin, monellin, thaumatin, curculin, brazzein, mabinlin), D-amino acids (such as D-phenylalanine, D-tryptophan) or extracts or fractions obtained from natural sources containing these amino acids and/or proteins and the physiologically acceptable salts of these amino acids and/or proteins, particularly the sodium, potassium, calcium or ammonium salts thereof, steviolgylcoside, stevioside, mono-, di-, tri- or tetra-alpha-glycosylated steviosides or rebaudiosides, steviolbiosid, rebaudiosides, rebaudioside A, rebaudioside B, rebaudioside C, rebaudioside D, rebaudioside E, rebaudioside F, rebaudioside G, rebaudioside H, rebaudioside M, rebaudioside N, rebaudioside X, dulcoside, rubusoside, mono-, di-, tri- or tetra-alpha-glycosylated rubusosides, suavioside A, suavioside B, suavioside G, suavioside H, suavioside I, suavioside J, baiyunoside 1, baiyunoside 2, phlomisoside 1, phlomisoside 2, phlomisoside 3, phlomisoside 4, abrusoside A, abrusoside B, abrusoside C, abrusoside D, cyclocaryoside A, cyclocaryoside I, oslandin, polypodoside A, strogin 1, strogin, 2, strogin 4, selligueanin A, dihydroquercetin-3-acetate, perillartin, telosmosid A15, periandrin I-V, pterocaryoside, cyclocaryoside, mukurozioside, trans-anethol, bryoside, bryonoside, bryonodulcoside, carnosifloside, scandenoside, gypenoside, hematoxylin, cyanin, chlorogenic acid, albiziasaponin, telosmoside, gaudichaudiosid, balansin A, balansin B, mogrosides, such as mogroside V, hernandulcine, monatin, glycyrrhetinic acid and its derivatives, particularly glycyrrhizin, preferably glycyrrhizin ammonium salt, extracts or enriched fractions of such extracts such as extracts of Thaumatococcus or Stevia ssp., particularly Stevia rebaudiana, stevia leaf extract, swingle extracts, particularly Momordica or Siratia grosvenorii or LuoHan-Guo, extracts of Glycerrhyzia ssp., particularly Glycerrhyzia glabra or Glycerrhyzia uralensis, extracts of Rubus ssp., particularly Rubus suavissimus or Rubus chingii comprising rubusoside, extracts of Mycetia balansae, preferably comprising balansin A and/or balansin B,
      synthetic sweet tasting substances, preferably selected from the group consisting of magap, sodium cyclamate or other physiologically acceptable salts of cyclamic acid, acesulfam K, saccharin, saccharin sodium salt, aspartam, superaspartam, neotam, alitam, advantam, perillartin, sucralose, lugduname, carrelame, sucrononate and sucrooctate.

Particularly preferably, the, one, two, three or more or all sweet tasting substance(s) is/are selected from the group consisting of sucrose, fructose, glucose, steviosides, rebaudiosides, rebaudioside A, rebaudioside M, mono-, di-, tri- or tetra-alpha-glycosylated steviosides or rebaudiosides, rubusoside, mono-, di-, tri- or tetra-alpha-glycosylated rubusosides.

Preferably, the method according to the invention further comprises the step
b.2) providing one or more sweet taste modulating substance(s) selected from the group consisting of hesperetin, hesperetin dihydrochalcone, naringenin, phloretin, eriodictyol, homoeriodictyol, phyllodulcin, neohesperidindihydrochalkon, naringindihydrochalkon, phloretin, extracts of Hydrangea macrophylla ssp. serrata, in particular var. Oamacha, Amacha or Amagi amacha comprising active amounts of phyllodulcin,
wherein in step c) the compound(s) provided in step a), the one or more sweet tasting substance(s) provided in step b), and the one or more sweet taste modulating substance(s) provided in step b.2) are mixed. In this case, the provided components may be mixed simultaneously or subsequently. For example, the compound(s) provided in step a) and the sweet tasting substance(s) provided in step b) may be mixed first and then be mixed with the sweet taste modulating substance(s) provided in step b.2). Furthermore, in a subsequent mixing, as described herein, mixing of the provided components may be in any order of the components.

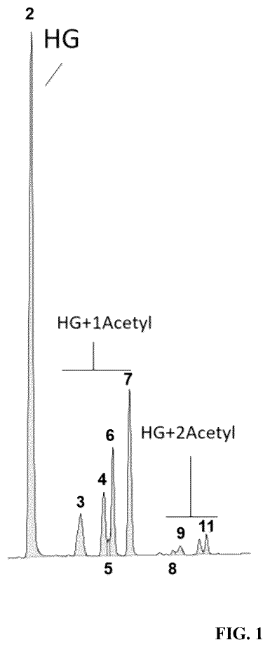

FIG. 1 describes the LC-HRMS chromatogram of acetylated products of HG obtained in Example 1. Charged aerosol detector signal is shown.

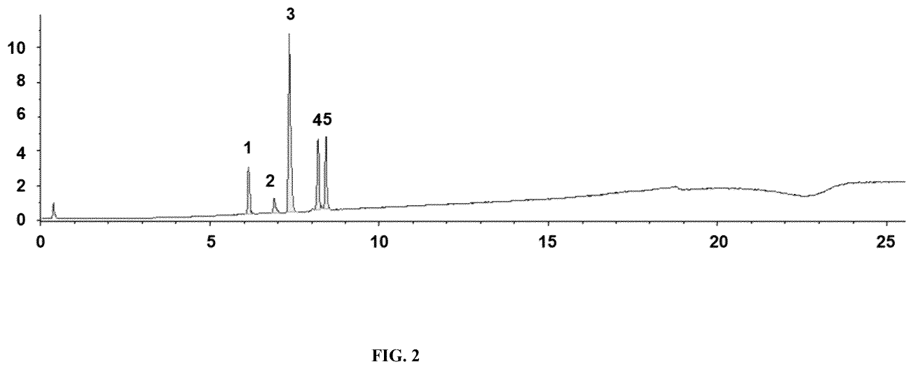

FIG. 2 describes the LC-HRMS chromatogram of acetylated products of HG obtained in Example 2. Charged aerosol detector signal is shown. Peak 3 corresponds to compound no. 9.

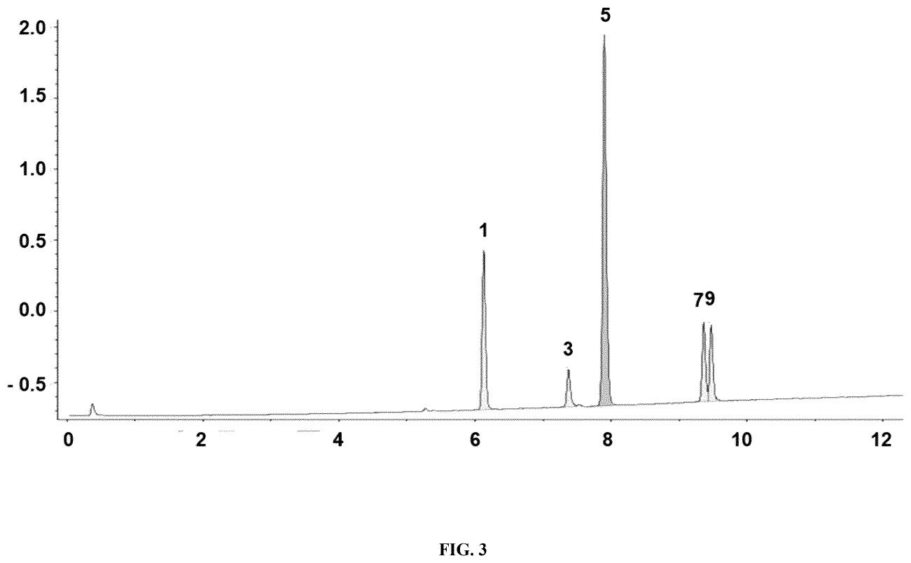

FIG. 3 describes the LC-HRMS chromatogram of acetylated products of HG obtained in Example 3. Charged aerosol detector signal is shown. Peak 3 corresponds to compound no. 25.

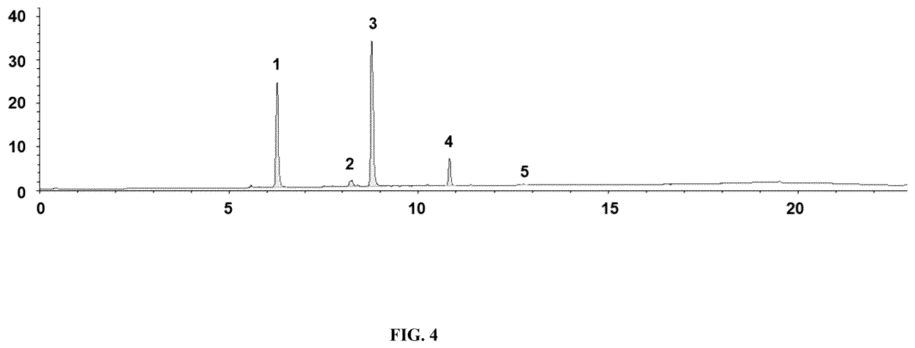

FIG. 4 describes the LC-HRMS chromatogram of acetylated products of HG obtained in Example 3. Charged aerosol detector signal is shown. Peak 3 corresponds to compound no. 41.

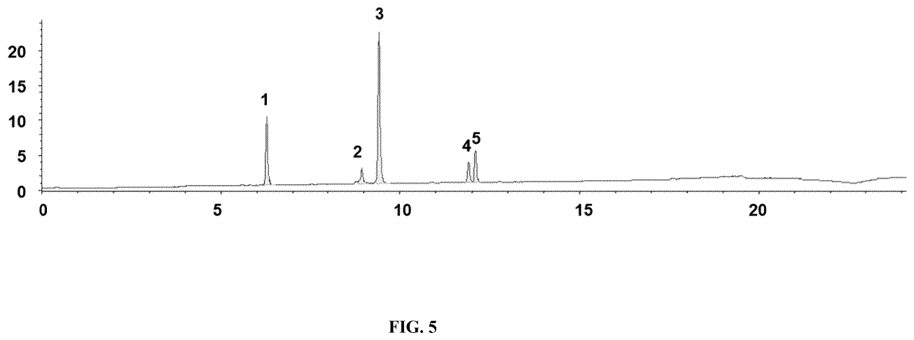

FIG. 5 describes the LC-HRMS chromatogram of acetylated products of HG obtained in Example 3. Charged aerosol detector signal is shown. Peak 3 corresponds to compound no. 57.

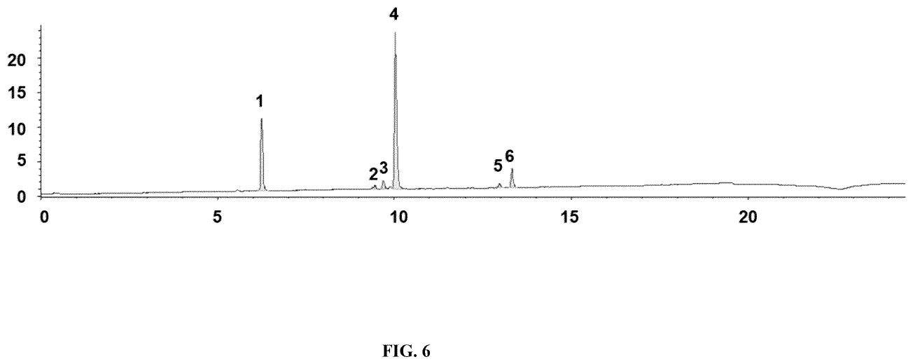

FIG. 6 describes the LC-HRMS chromatogram of acetylated products of HG obtained in Example 3. Charged aerosol detector signal is shown. Peak 3 corresponds to compound no. 73.

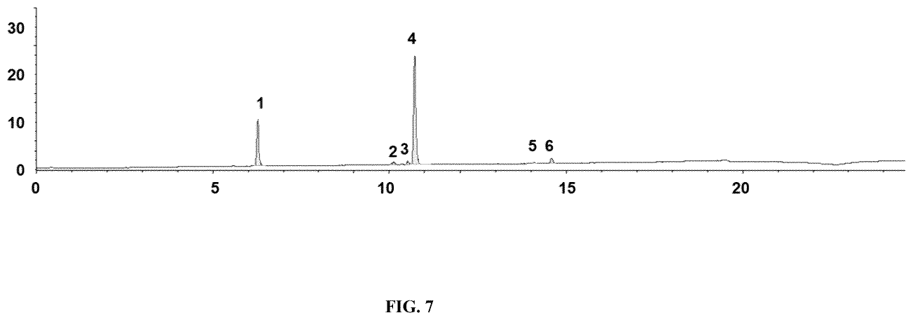

FIG. 7 describes the LC-HRMS chromatogram of acetylated products of HG obtained in Example 3. Charged aerosol detector signal is shown. Peak 3 corresponds to compound no. 89.

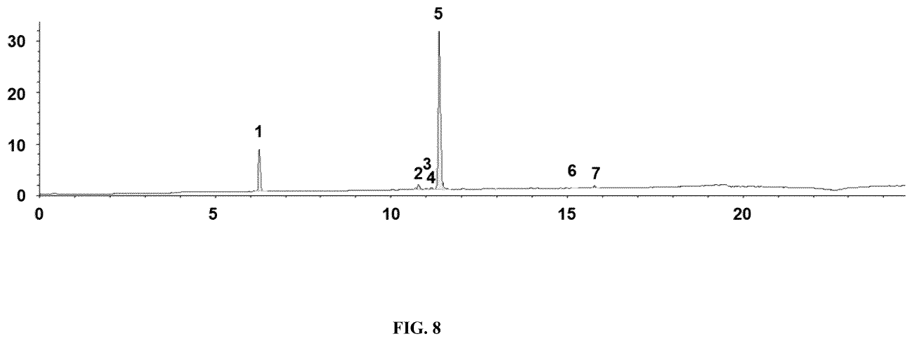

FIG. 8 describes the LC-HRMS chromatogram of acetylated products of HG obtained in Example 3. Charged aerosol detector signal is shown. Peak 3 corresponds to compound no. 105.

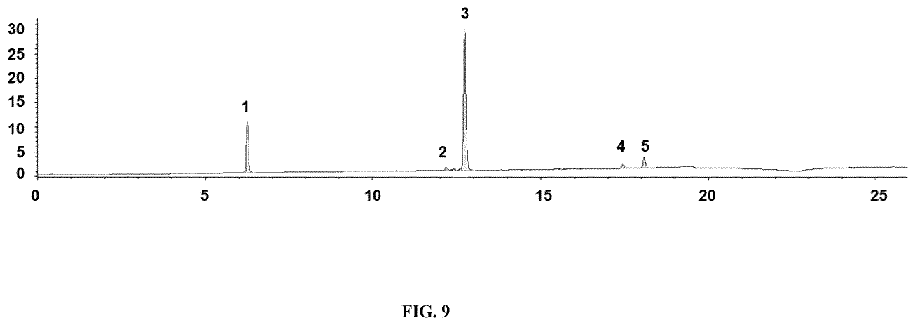

FIG. 9 describes the LC-HRMS chromatogram of acetylated products of HG obtained in Example 3. Charged aerosol detector signal is shown. Peak 3 corresponds to compound no. 137.

Further aspects and advantages of the invention result from the subsequent description of preferred examples.

### EXAMPLES

### Example 1: Chemical Acylation of HG

500 mg hesperetin dihydrochalcone-4′-O-β-d-glucoside (HG) were dissolved in 10 mL tetrahydrofuran before 100 μL acetyl acetate and 0.15 μL triethylamine were added at room temperature. The solution was stirred at room temperature for 18 h. The reaction was quenched with 15 mL H₂O and the aqueous layer was extracted with ethyl acetate (3×10 mL). The combined organic layers were dried over Na₂SO₄ and the solvent was removed under reduced pressure.

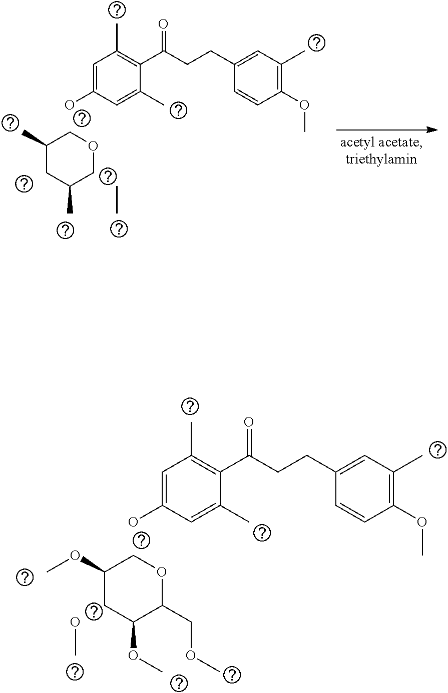

The obtained compounds were analysed by LC-HRMS (results shown in FIG. 1).

### Example 2: Enzymatic Acylation of HG

Hesperetin dihydrochalcone-4′-O-β-d-glucoside (HG) was resuspended in triacetin, ethyl acetate, acetone or acetonitril at concentrations of 5 g/L and dissolved via incubation at 70° C. Lipase Novozym 435 (Novozymes, Lyngby, Denmark) was added to the solutions at concentrations of 10 g/L and in case of acetonitrile or acetone solvent, ethyl acetate was added at a concentration of 3 g/L. The solutions were incubated under agitation for 4.5 h at 70° C. After incubation, the lipase was removed via filtration. The samples were analysed via HPLC-MS (results shown in FIG. 2) including the following results:

| | | RT | | Area |
|---|---|---|---|---|
| | Peak | [min] | Area | Frac. % |
| MW 466: HG | 1 | 6.17 | 10.5354 | 10.79 |
| MW 508: Acetyl-HG | 2 | 6.92 | 4.5623 | 4.67 |
| MW 508: compound 9 | 3 | 7.37 | 49.1216 | 50.30 |
| MW 550: Diacetyl-HG | 4 | 8.22 | 17.0567 | 17.47 |
| MW 550: Diacetyl-HG | 5 | 8.45 | 16.3841 | 16.78 |

### Example 3: Enzymatic Acylation of HG

Hesperetin dihydrochalcone-4′-O-β-d-glucoside (HG) was resuspended in acetone or acetonitril at concentrations of 5 g/L and dissolved via incubation at 70° C. Lipase Novozym 435 (Novozymes, Lyngby, Denmark) was added to the solutions at concentrations of 10 g/L and an acyl donor of the group of ethyl acetate, ethyl propionate, ethyl butyrate, ethyl pentanoate, ethyl hexanoate, ethyl heptanoate, ethyl octanoate, ethyl nonanoate or ethyl decanoate was added at a concentration of 3, 3.5, 4, 4.5, 5, 5.5, 6, 6.5 or 7 g/L, respectively.

The solutions were incubated under agitation for 4.5 h at 70° C. After incubation, the lipase was removed via filtration. The samples were analysed via HPLC-MS (results shown in FIGS. 3 to 9) including the following results (peaks classified as n.d. were below the threshold and could not be determined):

| Propanoyl donor such as | | RT | | Area |
|---|---|---|---|---|
| ethyl propanoate, FIG. 3 | Peak | [min] | Area | Frac. % |
| MW 466: HG | 1 | 6.15 | 15.1661 | 20.614 |
| MW 522: Propanoyl-HG | 3 | 7.39 | 3.9652 | 5.390 |
| MW 522: compound 25 | 5 | 7.92 | 39.5350 | 53.736 |
| MW 578: Di-Propanoyl-HG | 7 | 9.38 | 7.5235 | 10.226 |
| MW 578: Di-Propanoyl-HG | 9 | 9.49 | 7.3826 | 10.034 |

| Butanoyl donor such as | | RT | | Area |
|---|---|---|---|---|
| ethyl butanoate, FIG. 4 | Peak | [min] | Area | Frac. % |
| MW 466: HG | 1 | 6.29 | 107.8425 | 34.63 |
| MW 536: Butanoyl-HG | 2 | 8.26 | 8.7268 | 2.80 |
| MW 536: compound 41 | 3 | 8.78 | 167.2373 | 53.71 |
| MW 606: Di-Butanoyl-HG | 4 | 10.83 | 26.0726 | 8.37 |
| MW 606: Di-Butanoyl-HG | 5 | 12.78 | 1.4994 | 0.48 |

| Pentanoyl donor such as | | RT | | Area |
|---|---|---|---|---|
| ethyl pentanoate, FIG. 5 | Peak | [min] | Area | Frac. % |
| MW 466: HG | 1 | 6.30 | 38.739 | 22.00 |
| MW 550: Pentanoyl-HG | 2 | 8.95 | 10.594 | 6.02 |
| MW 550: compound 57 | 3 | 9.43 | 95.826 | 54.42 |
| MW 634: Di-Pentanoyl-HG | 4 | 11.93 | 12.129 | 6.89 |
| MW 634: Di-Pentanoyl-HG | 5 | 12.12 | 18.791 | 10.67 |

| Hexanoyl donor such as | | RT | | Area |
|---|---|---|---|---|
| ethyl hexanoate, FIG. 6 | Peak | [min] | Area | Frac. % |
| MW 466: HG | 1 | 6.27 | 43.3261 | 25.79 |
| n.d. | 2 | 9.47 | 2.8629 | 1.70 |
| MW 564: Hexanoyl-HG | 3 | 9.71 | 5.2846 | 3.15 |
| MW 564: compound 73 | 4 | 10.05 | 102.0720 | 60.76 |
| MW 662: Di-Hexanoyl-HG | 5 | 12.98 | 2.8740 | 1.71 |
| MW 662: Di-Hexanoyl-HG | 6 | 13.35 | 11.5747 | 6.89 |

| Heptanoyl donor such as | | RT | | Area |
|---|---|---|---|---|
| ethyl heptanoate, FIG. 7 | Peak | [min] | Area | Frac. % |
| MW 466: HG | 1 | 6.29 | 40.5311 | 25.64 |
| n.d. | 2 | 10.14 | 3.1650 | 2.00 |
| MW 578: Heptanoyl-HG | 3 | 10.54 | 2.8867 | 1.83 |
| MW 578: compound 89 | 4 | 10.73 | 105.6514 | 66.83 |
| MW 690: Di-Heptanoyl-HG | 5 | 14.12 | 0.9876 | 0.62 |
| MW 690: Di-Heptanoyl-HG | 6 | 14.60 | 4.8688 | 3.08 |

| Octanoyl donor such as | | RT | | Area |
|---|---|---|---|---|
| ethyl octanoate, FIG. 8 | Peak | [min] | Area | Frac. % |
| MW 466: HG | 1 | 6.27 | 34.0077 | 17.97 |
| n.d. | 2 | 10.79 | 4.8576 | 2.57 |
| MW 592: compound 105 | 5 | 11.38 | 147.4929 | 77.94 |
| MW 718: Di-Octanoyl-HG | 6 | 15.20 | 0.8643 | 0.46 |
| MW 718: Di-Octanoyl-HG | 7 | 15.79 | 2.0192 | 1.07 |

| Decanoyl donor such as | | RT | | Area |
|---|---|---|---|---|
| ethyl decanoate, FIG. 9 | Peak | [min] | Area | Frac. % |
| MW 466: HG | 1 | 6.28 | 43.4466 | 21.00 |
| n.d. | 2 | 12.20 | 2.9565 | 1.43 |
| MW 620: compound 137 | 3 | 12.75 | 146.1653 | 70.66 |
| n.d. | 4 | 17.48 | 3.7992 | 1.84 |
| MW 774: Di-Decanoyl-HG | 5 | 18.10 | 10.4956 | 5.07 |

### Example 4: Sweet Taste of Compounds of Formula (I)

Compounds 9, 41, 57, 73 and 89 were provided as described in Example 3 and purified to >95% with preparative chromatography. The obtained compounds were used for sensory evaluations.

The intrinsic sweetness of the compounds was determined by a panel of 20 trained panellists. The intrinsic sweetness of a compound is determined by evaluating the sweetness of the compound at different concentrations in water against a 1.5% sucrose solution. It is then determined, which concentration does not provide a statistical significant difference (p<0.05) between the sample and the 1.5% sucrose control (in both directions: sweeter or less sweet), i.e. the concentration of the respective compound, which provides equal sweetness as the 1.5% sucrose solution.

As shown in the table below, the concentrations of the acylated compounds, which represent equal sweetness as the 1.5% sucrose solution, are approximately ⅓ to 1/17 of the required concentration of HG (non-acylated). Thus, acylation of HG provides a strong increase of the intrinsic sweetness.

| | Concentration of equal sweetness |
|---|---|
| Compound | as 1.5% sucrose control [mg/kg] |
| HG (non-acylated | 50 |
| control) | |
| 41 | 5 |
| 57 | 3 |
| 73 | 5 |
| 89 | 5 |
| 9 | 15 |

### Example 5: Sweet Taste of a Mixture of Compounds of Formula (I)

A mixture of butanoylated HG and a mixture of pentanoylated HG were provided as described in Example 3.

The intrinsic sweetness was determined as described in Example 4. The results are shown in the subsequent table. As shown in the table, the concentrations of the acylated compounds, which represent equal sweetness as the 1.5% sucrose solution, are approximately ⅕ to ⅛ of the required concentration of HG (non-acylated). Thus, acylation of HG provides a strong increase of the intrinsic sweetness.

| | Concentration of equal sweetness |
|---|---|
| Compound | as 1.5% sucrose control [mg/kg] |
| Butanoylated mixture | 10 |
| (53% compound 41; see | |
| Example 3 and FIG. 4) | |
| Pentanoylated mixture | 6 |
| (54% compound 57; see | |
| Example 3 and FIG. 5) | |

### Example 6: Modulating the Sweet Taste of a Sweet Tasting Substance (Sucrose)

A 5% sucrose solution was prepared. Compounds 9, 41, 57, 73 and 89 were provided as described in Example 3 and purified to >95% with preparative chromatography.

The sensory profile of the sucrose solution (control) and the sucrose solution including HG (control), compounds 9, 41, 57, 73 or 89 was determined by a trained flavourist panel comparing the impact, intensity and mouthfeel.

It was found that the addition of HG increases the impact, intensity and mouthfeel. Further, it was found that much lower concentrations of acylated HG are required to provide a comparable effect:

| | | | | | Lingering |
|---|---|---|---|---|---|
| | | Impact | Intensity | Mouthfeel | sweetness |
| A1 | 5% sucrose solution | 3 | 4 | 4 | 1 |
| A2 | A1 + HG (50 ppm) | 4 | 5 | 4-5 | 2 |
| A3 | A1 + compound 9 | 3 | 4-5 | 4-5 | 2 |
| | (15 ppm) | | | | |
| A4 | A1 + compound 41 | 4-5 | 5-6 | 5 | 2-3 |
| | (5 ppm) | | | | |
| A5 | A1 + compound 57 | 4-5 | 5-6 | 5 | 3 |
| | (3 ppm) | | | | |
| A6 | A1 + compound 73 | 4-5 | 6-7 | 5 | 3-4 |
| | (5 ppm) | | | | |
| A7 | A1 + compound 89 | 4-5 | 6-7 | 5 | 3-4 |
| | (5 ppm) | | | | |

### Example 7: Modulating the Sweet Taste of a Sweet Tasting Substance (Sucrose)

The sensory impact of the compounds 9, 41, 57, 73 and 89 on a 5% sucrose solution in water was analysed by a trained sensory panel (n=20) using a series of paired comparison tests (Duo test). The samples were coded and randomized.

It was found that the tested compounds are able to increase the sweetness of the sucrose solution. Surprisingly, the observed effects were similar to those observed for HG, however, wherein the compounds according to formula (I) were applied in much lower concentrations.

| | Dosage | Increase of | |
|---|---|---|---|
| Compound | [mg/kg] | sweetness | t-test |
| HG | 50 | 34.6 | 0.001 |
| Compound 9 | 10 | 33.1 | 0.007 |
| Compound 41 | 5 | 24.4 | 0.0054 |
| Compound 57 | 3 | 25.2 | 0.0070 |
| Compound 73 | 5 | 23.1 | 0.0291 |
| Compound 89 | 5 | 27.7 | 0.0087 |

### Example 8: Modulating the Sweet Taste of Sweet Tasting Substances

A sugar base (5% sucrose+0.1% citric acid) was provided. Further, the sweet tasting substances RS (20% Rubusoside), SG (90% steviol oligoglucosides), HC (8% Hesperetin dihydrochalcone), LHG (52% mogroside V) and the sweet taste modulating substances PD (>95% Phyllodulcin), HT (>85% Hesperetin) and PH (>98% Phloretin) were provided and added to the sugar base to produce different samples (A).

A dose of 2 mg/kg compound 41 or, respectively, 1.2 mg/kg compound 57 was added to the samples to obtain samples (B).

Different taste descriptors were compared between the respective samples (A) and (B) by a panel of five flavorists (n=5) using a scale of 0-9.

The panelists were asked to neutralize with tap water between each sample and the intensity of the descriptors of each sample were defined before tasting the next sample. The tasting of each RS, SG, HC, LHG, PD, HT or PH (samples (A)) were followed by the samples with added RS, SG, HC, LHG, PD, HT or PH+compound (41 or 57) (samples (B)) with each being compared to the intensities of the base.

### Example 8.1: Compound 41

### Descriptor “Onset Sweetness”:

| | Dose | Onset sweetness (0-9) | | |
|---|---|---|---|---|
| Sample | [mg/kg] | A | B | Δ |
| Base | | 2.0 | 2.5 | 0.5 |
| PH | 25 | 3.0 | 3.0 | 0.0 |
| HT | 12 | 2.5 | 3.0 | 0.5 |
| HC | 50 | 3.0 | 4.0 | 1.0 |
| RS | 75 | 2.5 | 3.5 | 1.0 |
| LHG | 20 | 2.0 | 3.0 | 1.0 |
| PD | 3 | 2.5 | 3.5 | 1.0 |
| SG | 60 | 3.0 | 4.5 | 1.5 |

| | Dose | Overall sweetness (0-9) | | |
|---|---|---|---|---|
| Sample | [mg/kg] | A | B | Δ |
| Base | | 3.0 | 4.0 | 1.0 |
| PH | 25 | 4.0 | 4.5 | 0.5 |
| HT | 12 | 3.5 | 4.0 | 0.5 |
| HC | 50 | 4.0 | 5.0 | 1.0 |
| RS | 75 | 3.5 | 5.0 | 1.5 |
| LHG | 20 | 3.5 | 4.5 | 1.0 |
| PD | 3 | 4.0 | 4.5 | 0.5 |
| SG | 60 | 4.0 | 5.5 | 1.5 |

| | Dose | Mouthfeel (0-9) | | |
|---|---|---|---|---|
| Sample | [mg/kg] | A | B | Δ |
| Base | | 3.0 | 4.0 | 1.0 |
| PH | 25 | 4.0 | 4.5 | 0.5 |
| HT | 12 | 3.5 | 4.0 | 0.5 |
| HC | 50 | 4.0 | 5.0 | 1.0 |
| RS | 75 | 3.5 | 5.0 | 1.5 |
| LHG | 20 | 3.5 | 4.5 | 1.0 |
| PD | 3 | 4.0 | 4.5 | 0.5 |
| SG | 60 | 4.0 | 5.5 | 1.5 |

| | Dose | Lingering sweetness (0-9) | | |
|---|---|---|---|---|
| Sample | [mg/kg] | A | B | Δ |
| Base | | 0.0 | 1.0 | 1.0 |
| PH | 25 | 0.0 | 1.0 | 1.0 |
| HT | 12 | 0.0 | 1.0 | 1.0 |
| HC | 50 | 0.5 | 1.0 | 0.5 |
| RS | 75 | 0.0 | 0.5 | 0.5 |
| LHG | 20 | 0.0 | 0.5 | 0.5 |
| PD | 3 | 1.0 | 2.0 | 1.0 |
| SG | 60 | 1.0 | 2.0 | 1.0 |

| | Dose | Onset sweetness (0-9) | | |
|---|---|---|---|---|
| Sample | [mg/kg] | A | B | Δ |
| Base | | 2.0 | 2.5 | 0.5 |
| PH | 25 | 2.5 | 3.0 | 0.5 |
| HT | 12 | 2.5 | 3.5 | 1.0 |
| HC | 50 | 2.5 | 3.5 | 1.0 |
| RS | 75 | 2.5 | 3.5 | 1.0 |
| LHG | 20 | 2.0 | 2.5 | 0.5 |
| PD | 3 | 2.5 | 3.0 | 0.5 |
| SG | 60 | 3.0 | 3.5 | 0.5 |

| | Dose | Overall sweetness (0-9) | | |
|---|---|---|---|---|
| Sample | [mg/kg] | A | B | Δ |
| Base | | 3.0 | 4.0 | 1.0 |
| PH | 25 | 3.5 | 4.5 | 1.0 |
| HT | 12 | 3.5 | 5.0 | 1.5 |
| HC | 50 | 3.5 | 4.5 | 1.0 |
| RS | 75 | 3.5 | 5.0 | 1.5 |
| LHG | 20 | 3.5 | 4.5 | 1.0 |
| PD | 3 | 4.0 | 5.0 | 1.0 |
| SG | 60 | 4.0 | 4.5 | 0.5 |

### Descriptor “Mouthfeel”:

| | Dose | Mouthfeel (0-9) | | |
|---|---|---|---|---|
| Sample | [mg/kg] | A | B | Δ |
| Base | | 3.0 | 3.5 | 0.5 |
| PH | 25 | 3.5 | 4.0 | 0.5 |
| HT | 12 | 3.5 | 4.0 | 0.5 |
| HC | 50 | 3.5 | 4.0 | 0.5 |
| RS | 75 | 4.0 | 5.0 | 1.0 |
| LHG | 20 | 3.0 | 4.0 | 1.0 |
| PD | 3 | 3.5 | 4.5 | 1.0 |
| SG | 60 | 3.5 | 4.5 | 1.0 |

### Descriptor “Lingering Sweetness”:

| | Dose | Lingering sweetness (0-9) | | |
|---|---|---|---|---|
| Sample | [mg/kg] | A | B | Δ |
| Base | | 0.0 | 1.0 | 1.0 |
| PH | 25 | 0.0 | 1.5 | 1.5 |
| HT | 12 | 0.0 | 2.0 | 2.0 |
| HC | 50 | 0.0 | 2.0 | 2.0 |
| RS | 75 | 0.0 | 1.0 | 1.0 |
| LHG | 20 | 0.0 | 1.0 | 1.0 |
| PD | 3 | 1.0 | 1.5 | 0.5 |
| SG | 60 | 1.0 | 1.5 | 0.5 |

### Example 9: Sweet Taste Modulating Properties of the Compounds in Lemonade Bases with Reduced Sugar Content

A dose of 5 mg/kg compound 41 or, respectively, 3 mg/kg compound 57 were applied to different sugar reduced lemonade bases to restore the overall and sweet taste of the respective matrix with reduced sugar content. Different descriptors were determined by a panel of five flavorists (n=5) and given as intensities from 0-9.

The lemonade bases were “full sugar”, “sugar reduced”, “Stevia Hybride”, “Sugar free base Suc/Ace K” and “Sugar free base Asp/Ace K” with the following recipes:

| | | | | Sugar | Sugar |
|---|---|---|---|---|---|
| | Full | Sugar | Stevia | free base | free base |
| Ingredient | sugar | reduced | Hybride | Suc/Ace K | Asp/Ace K |
| Invert sugar | 126.00 | 100.80 | 63.00 | | |
| Rebaudioside A | | | 0.12 | | |
| Sucralose | | | | 0.07 | |
| Aspartame | | | | | 0.12 |
| Acesulfame K | | | | 0.04 | 0.08 |
| Citric acid | 2.50 | 2.50 | 2.50 | 2.50 | 2.50 |
| Tri Sodium citrate | 0.30 | 0.30 | 0.30 | 0.30 | 0.30 |
| Lemon flavour | 0.60 | 0.60 | 0.60 | 0.60 | 0.60 |
| Water | 870.60 | 895.80 | 933.48 | 996.49 | 996.40 |

### Lemonade Base “Sugar Reduced”:

| Test | Dosage | | Overall | | Lingering |
|---|---|---|---|---|---|
| solution | [mg/kg] | Onset | sweetness | Mouthfeel | sweetness |
| Full sugar | | 4.5 | 5 | 4.5 | 0 |
| Sugar | | 3 | 3.5 | 3 | 0 |
| reduced | | | | | |
| base | | | | | |
| Sugar | 5 | 4 | 4.5 | 3.5 | 0.5 |
| reduced | | | | | |
| base + | | | | | |
| compound 41 | | | | | |
| Sugar | 3 | 4 | 4.5 | 4 | 2 |
| reduced | | | | | |
| base + | | | | | |
| compound 57 | | | | | |

Lemonade base “Stevia Hybrid”:

| | Dosage | | Overall | | Lingering |
|---|---|---|---|---|---|
| Test solution | [mg/kg] | Onset | sweetness | Mouthfeel | sweetness |
| Full sugar | | 4.5 | 5 | 4.5 | 0 |
| Stevia Hybrid | | 3.5 | 4.5 | 3 | 0.5 |
| base | | | | | |
| Stevia Hybrid | 5 | 4 | 5.5 | 4.5 | 1 |
| base + | | | | | |
| compound | | | | | |
| 41 | | | | | |
| Stevia Hybrid | 3 | 4 | 6 | 5 | 2.5 |
| base + | | | | | |
| compound | | | | | |
| 57 | | | | | |

### Lemonade Base “Sugar Free Base Suc/Ace K”:

| | Dosage | | Overall | | Lingering |
|---|---|---|---|---|---|
| Test solution | [mg/kg] | Onset | sweetness | Mouthfeel | sweetness |
| Full sugar | | 4.5 | 5 | 4.5 | 0 |
| Sugar free base | | 2 | 3.5 | 1.5 | 2 |
| Suc/Ace K | | | | | |
| Sugar free base | 5 | 2.5 | 4.5 | 2 | 3 |
| Suc/Ace K + | | | | | |
| compound 41 | | | | | |
| Sugar free base | 3 | 3 | 5 | 2.5 | 4 |
| Suc/Ace K + | | | | | |
| compound 57 | | | | | |

### Lemonade Base “Sugar Free Base Asp/Ace K”:

| | Dosage | | Overall | | Lingering |
|---|---|---|---|---|---|
| Test solution | [mg/kg] | Onset | sweetness | Mouthfeel | sweetness |
| Full sugar | | 4.5 | 5 | 4.5 | 0 |
| Sugar free base | | 3 | 4 | 2.5 | 2 |
| Asp/Ace K | | | | | |
| Sugar free base | 5 | 4 | 5 | 3.5 | 2.5 |
| Asp/Ace K + | | | | | |
| compound 41 | | | | | |
| Sugar free base | 3 | 3.5 | 5 | 3.5 | 3.5 |
| Asp/Ace K + | | | | | |
| compound 57 | | | | | |

### APPLICATION EXAMPLES

### Application Example 1

### Spray-Dried Preparation as a Semi-Finished Product for Flavouring of Finished Products

| Ingredient | Use in % by weight | | | | | | |
|---|---|---|---|---|---|---|---|
| Preparation | A | B | C | D | E | F | G |
| Drinking water | 60.80 | 60.80 | 60.80 | 60.80 | 60.80 | 60.80 | 60.80 |
| Maltodextrin from | 31.50 | 29.70 | 28.80 | 27.00 | 30.20 | 30.00 | 31.50 |
| wheat | | | | | | | |
| Gum Arabic | 6.10 | 6.10 | 6.10 | 6.10 | 6.10 | 6.10 | 6.10 |
| 6″-O-Butanoyl-HG, | 0.40 | 0.36 | 0.26 | 0.25 | 0.18 | 0.18 | — |
| compound 41 | | | | | | | |
| 6″-O-Pentanoyl-HG, | — | — | — | 0.15 | — | — | 0.24 |
| compound 73 | | | | | | | |
| Hesperetin | — | 2.20 | — | — | 1.10 | 1.10 | — |
| Phloretin | — | — | 3.30 | — | — | — | — |
| Hesperetindihydrochalkon | — | — | — | — | 0.50 | — | — |
| Phyllodulcin (dry | — | — | — | — | — | 0.70 | — |
| Oamacha extract | | | | | | | |
| containing 70% | | | | | | | |
| Phyllodulcin) | | | | | | | |
| Ingredient | Use in % by weight | | | | | | |
| Preparation | H | I | J | K | L | M | |
| Drinking water | 60.80 | 60.80 | 60.80 | 60.80 | 60.80 | 60.80 | |
| Maltodextrin from | 29.70 | 28.80 | 27.00 | 30.20 | 30.00 | 29.70 | |
| wheat | | | | | | | |
| Gum Arabic | 6.10 | 6.10 | 6.10 | 6.10 | 6.10 | 6.10 | |
| 6″-O-Butanoyl-HG, | — | — | — | — | 1.00 | — | |
| compound 41 | | | | | | | |
| 6″-O-Pentanoyl-HG, | 0.22 | 0.16 | 0.15 | 0.11 | — | 0.60 | |
| compound 73 | | | | | | | |
| Hesperetin | 2.20 | — | 1.10 | 1.10 | | — | |
| Phloretin | — | 3.30 | — | — | | — | |
| Hesperetindihydrochalkon | — | — | 0.50 | — | | — | |
| Phyllodulcin (dry | — | — | — | 0.70 | | — | |
| Oamacha extract | | | | | | | |
| containing 70% | | | | | | | |
| Phyllodulcin) | | | | | | | |

The drinking water is placed in a container and maltodextrin and gum arabic is dissolved in it. Then the flavouring is emulsified in the carrier solution with a Turrax. The temperature of the spray solution should not exceed 30° C. The mixture is then spray-dried (inlet nominal temperature: 185-195° C. outlet nominal temperature: 70-75° C.).

### Application Example 2

Combination with Sweeteners

90 g sucrose and 10 g tagatose are added to 0.5 g of a spray-dried semi-finished product from application example 1 (according to preparation A, D or G) and mixed. The product can for example be used as a sweetener.

### Application Example 3

### Chewing Gum

| Part | Ingredient | % b.w. |
|---|---|---|
| A | Chewing gum base from “Jagum T” company | 30.90 |
| B | Sorbitol, powdered | 39.00 |
| | Isomalt ® (Palatinit GmbH) | 9.50 |
| | Xylitol | 2.00 |
| | Mannitol | 3.00 |
| | Rebaudioside A 98% | 0.20 |
| | Emulgum ® (Colloides Naturels, Inc.) | 0.30 |
| C | Sorbitol, 70% | 14.00 |
| | Glycerin | 1.00 |
| D | Flavouring, containing 6″-O-Butanoyl-HG, | 0.10 |
| | compound (41), and/or 6″-O-Pentanoyl-HG, | |
| | compound (73), based on the total weight of | |
| | the flavouring (preparation A, D or G from | |
| | Application Example 1, respectively) | |

Parts A to D are mixed and kneaded intensively. The raw mass can be processed by way of example in the form of thin strips into ready-to-consume chewing gum.

### Application Example 4

### Toothpaste

| Part | Ingredient | % b.w. |
|---|---|---|
| A | Demineralized water | 21.00 |
| | Sorbitol (70%) | 46.00 |
| | Solbrol ® M, sodium salt (Bayer AG, | 0.15 |
| | p-hydroxybenzoic acid alkyl ester) | |
| | Trisodium phosphate | 0.10 |
| | Rebaudioside A, 98% | 0.10 |
| | Sodium monofluorophosphate | 1.12 |
| | Polyethylene glycol 1500 | 5.00 |
| B | Sident 9 (abrasive silicon dioxide) | 10.00 |
| | Sident 22 S (thickening silicon dioxide) | 8.00 |
| | Sodium carboxymethylcellulose | 0.90 |
| | Titanium dioxide | 0.50 |
| C | Demineralized water | 4.63 |
| | Sodium lauryl sulfate | 1.50 |
| D | Flavouring, containing 0.04% 6″-O-Butanoyl-HG, | 1.00 |
| | compound (41), or 0.024% 6″-O-Pentanoyl-HG, | |
| | compound (73), based on the total weight of the | |
| | flavouring | |

The ingredients of parts A and B are in each case pre-mixed separately and stirred well under a vacuum at 25-30° C. for 30 minutes. Part C is pre-mixed and added to A and B; D is added and the mixture stirred well under a vacuum at 25-30° C. for 30 minutes. After pressure relief the toothpaste is finished and can be filled.

### Application Example 5

### Sugar-Free Hard Boiled Candy

| | Content (%) | | | | | | | | |
|---|---|---|---|---|---|---|---|---|---|
| Ingredient | A | B | C | D | E | F | G | H | I |
| Palatinite, Type M | 75.0 | 74.0 | 75.5 | 75.0 | 75.0 | 74.0 | 75.5 | 75.0 | 75.0 |
| Citric acid | — | 1.0 | 0.5 | — | — | 1.0 | 0.5 | — | — |
| Colouring, yellow | — | 0.0 | — | — | — | 0.0 | — | — | — |
| Colouring, red | — | — | 0.0 | — | — | — | 0.0 | — | — |
| Colouring, blue | 0.0 | — | — | 0.0 | 0.0 | — | — | 0.0 | 0.0 |
| Peppermint flavouring | 0.1 | — | — | 0.1 | 0.1 | — | — | 0.1 | 0.1 |
| Lemon flavouring | — | 0.1 | — | — | — | 0.1 | — | — | — |
| Red fruit flavouring | — | — | 0.1 | — | — | — | 0.1 | — | — |
| 6″-O-Butanoyl-HG, compound (41) | 0.0004 | 0.0002 | 0.0003 | 0.0004 | — | — | — | — | 0.0002 |
| 6″-O-Pentanoyl-HG, compound (73) | — | — | — | — | 0.0002 | 0.0001 | 0.0002 | 0.0002 | 0.0001 |
| Rebaudioside A 98% | — | 0.040 | — | 0.040 | — | 0.040 | — | 0.040 | — |
| hesperetin dihydrochalcone | — | — | 0.001 | — | — | — | 0.001 | — | — |
| Hesperetin | — | 0.001 | — | 0.001 | — | 0.001 | — | 0.001 | — |
| Phloretin | — | 0.002 | — | — | — | 0.002 | — | — | — |

| | |
|---|---|
| Water | ad 100 |

Palatinite was mixed with water and the mixture melted at 165° C. and then cooled to 115° C. The other ingredients were added and after mixing cast into moulds, following hardening removed from the moulds and then individually packaged.

### Application Example 6

### Sugar-Reduced Tomato Ketchup

A: Comparative preparation with sugar
B: Comparative preparation with reduced sugar content (compared to A)
C-H: Preparations according to the invention with reduced sugar content (compared to A) and 6″-O-Butanoyl-HG, compound (41)

| | Preparation (amounts in % by weight) | | | | | | | |
|---|---|---|---|---|---|---|---|---|
| Ingredient | A | B | C | D | E | F | G | H |
| Common salt | 2.00 | 2.00 | 2.00 | 2.00 | 2.00 | 2.00 | 2.00 | 2.00 |
| Starch, Farinex WM 55 | 1.00 | 1.00 | 1.00 | 1.00 | 1.00 | 1.00 | 1.00 | 1.00 |
| Sucrose | 12.00 | 9.60 | 9.20 | 8.40 | 9.60 | 9.60 | 8.40 | 8.40 |
| Tomato concentrate × 2 | 40.00 | 40.00 | 40.00 | 40.00 | 30.00 | 30.00 | 30.00 | 30.00 |
| Glucose syrup 80 Brix | 18.00 | 18.00 | 18.00 | 18.00 | 18.00 | 18.00 | 18.00 | 18.00 |
| Spirit vinegar 10% | 7.00 | 7.00 | 7.00 | 7.00 | 3.00 | 3.00 | 3.00 | 3.00 |
| 6″-O-Butanoyl-HG, | | | 0.06 | 0.05 | 0.05 | 0.05 | 0.03 | 0.04 |
| compound (41), 0.5% in | | | | | | | | |
| 1,2-propylene glycol | | | | | | | | |
| Hesperetin dihydrochal- | | | | 0.10 | | 0.05 | 0.05 | |
| cone (I) 0.5% in 1,2- | | | | | | | | |
| Propylene glycol | | | | | | | | |
| Hesperetin 2,5% in 1,2- | | | | | 0.10 | | 0.20 | |
| Propylene glycol | | | | | | | | |
| Phloretin 2,5% in 1,2- | | | | 0.20 | 0.20 | | | 0.20 |
| Propylene glycol | | | | | | | | |

| | |
|---|---|
| Water | ad 100 |

The ingredients are mixed in the stated sequence and the finished ketchup is homogenized using an agitator, poured into bottles and sterilized.

### Sugar-Reduced Tomato Ketchup

A: Comparative preparation with sugar
B: Comparative preparation with reduced sugar content (compared to A)
C-H: Preparations according to the invention with reduced sugar content (compared to A) and 6″-O-Pentanoyl-HG, compound (73)

| | Preparation (amounts in % by weight) | | | | | | | |
|---|---|---|---|---|---|---|---|---|
| Ingredient | A | B | C | D | E | F | G | H |
| Common salt | 2.00 | 2.00 | 2.00 | 2.00 | 2.00 | 2.00 | 2.00 | 2.00 |
| Starch, Farinex WM 55 | 1.00 | 1.00 | 1.00 | 1.00 | 1.00 | 1.00 | 1.00 | 1.00 |
| Sucrose | 12.00 | 9.60 | 9.20 | 8.40 | 9.60 | 9.60 | 8.40 | 8.40 |
| Tomato concentrate × 2 | 40.00 | 40.00 | 40.00 | 40.00 | 30.00 | 30.00 | 30.00 | 30.00 |
| Glucose syrup 80 Brix | 18.00 | 18.00 | 18.00 | 18.00 | 18.00 | 18.00 | 18.00 | 18.00 |
| Spirit vinegar 10% | 7.00 | 7.00 | 7.00 | 7.00 | 3.00 | 3.00 | 3.00 | 3.00 |
| 6″-O-Pentanoyl-HG, | | | 0.04 | 0.03 | 0.03 | 0.04 | 0.03 | 0.03 |
| compound (73), 0.5% in | | | | | | | | |
| 1,2-propylene glycol | | | | | | | | |
| Hesperetin dihydrochal- | | | | 0.10 | | 0.05 | 0.05 | |
| cone (I) 0.5% in 1,2- | | | | | | | | |
| Propylene glycol | | | | | | | | |
| Hesperetin 2.5% in 1,2- | | | | | 0.10 | | 0.20 | |
| Propylene glycol | | | | | | | | |
| Phloretin 2.5% in 1,2- | | | | 0.20 | 0.20 | | | 0.20 |
| Propylene glycol | | | | | | | | |

| | |
|---|---|
| Water | ad 100 |

The ingredients are mixed in the stated sequence and the finished ketchup is homogenized using an agitator, poured into bottles and sterilized.

### Application Example 7

### Reduced-Sugar Fruit Gums

| | | B ((% by | B ((% by |
|---|---|---|---|
| | | weight), | weight), |
| | | preparation | preparation |
| | A (% by | according | according |
| | weight), | to the | to the |
| | Comparative | present | present |
| Ingredient | preparation | invention | invention |
| Water | 23.70 | 25.60 | 25.60 |
| Saccharose | 34.50 | 8.20 | 8.20 |
| Glucose syrup, DE 40 | 31.89 | 30.09 | 30.09 |
| Iso Syrup C* Tru Sweet | 1.50 | 2.10 | 2.10 |
| 01750 (Cerestar GmbH) | | | |
| Gelatin 240 Bloom | 8.20 | 9.40 | 9.40 |
| Polydextrose (Litesse ® | — | 24.40 | 24.40 |
| Ultra, Danisco Cultor | | | |
| GmbH) | | | |
| Yellow and red colourings | 0.01 | 0.01 | 0.01 |
| Citric acid | 0.20 | | |
| Cherry flavouring, | — | 0.04 | — |
| containing 1% by weight | | | |
| of 6″-O- Butanoyl-HG, | | | |
| compound (41), based on | | | |
| the flavouring | | | |
| Cherry flavouring, | — | — | 0.02 |
| containing 1% by weight | | | |
| of 6″-O- Pentanoyl-HG, | | | |
| compound (73), based on | | | |
| the flavouring | | | |

Note:
Polydextrose is itself a non-sweet-tasting polysaccharide with a low calorific value.

### Application Example 8

### Carbonated Drink (Flavour Direction: Cola)

A: drink containing sugar (comparative) drink
B: low-calorie drink
C: low-calorie drink
D: low-calorie drink
E: low-calorie drink

| | A | B | C | D | E | F | G | H | I |
|---|---|---|---|---|---|---|---|---|---|
| | (% by | (% by | (% by | (% by | (% by | (% by | (% by | (% by | (% by |
| Ingredient | weight) | weight) | weight) | weight) | weight) | weight) | weight) | weight) | weight) |
| Phosphoric acid 85% | 0.64 | 0.64 | 0.64 | 0.64 | 0.64 | 0.64 | 0.64 | 0.64 | 0.64 |
| Citric acid, anhydrous | 0.06 | 0.06 | 0.06 | 0.06 | 0.06 | 0.06 | 0.06 | 0.06 | 0.06 |
| Caffeine | 0.06 | 0.06 | 0.06 | 0.06 | 0.06 | 0.06 | 0.06 | 0.06 | 0.06 |
| Sucrose | 63.60 | — | — | — | 12.90 | — | — | — | 12.90 |
| Sucralose | — | 0.13 | — | — | — | 0.13 | — | — | — |
| Erythritol | — | — | 6.00 | — | — | — | 6.00 | — | — |
| Aspartame | — | — | 0.35 | — | — | — | 0.35 | — | — |
| Rebaudioside A | — | — | — | — | 0.10 | — | — | | 0.10 |
| Rebaudioside M | — | — | — | — | 0.20 | — | — | — | 0.20 |
| Acesulfame K | — | — | 0.07 | | — | — | 0.07 | — | — |
| Sugar colouring | 0.80 | 0.80 | 0.80 | 0.80 | 0.80 | 0.80 | 0.80 | 0.80 | 0.80 |
| Cola type drink emulsion | 1.45 | 1.45 | 1.45 | 1.45 | 1.45 | 1.45 | 1.45 | 1.45 | 1.45 |
| Sodium benzoate | 0.11 | 0.11 | 0.11 | 0.11 | 0.11 | 0.11 | 0.11 | 0.11 | 0.11 |
| 6″-O-Butanoyl-HG, compound | — | 0.40 | 0.25 | 1.25 | 0.25 | | | | |
| (41), 0.5% in 1,2-propylene | | | | | | | | | |
| glycol | | | | | | | | | |
| 6″-O-Pentanoyl-HG, compound | — | — | — | — | — | 0.30 | 0.15 | 1.50 | 0.15 |
| (73), 0.5% in 1,2-propylene | | | | | | | | | |
| glycol | | | | | | | | | |

| | |
|---|---|
| Water | ad 100 |

The solid components or ingredients are individually mixed with water, combined and made up to 100 g with water. The concentrate obtained is then allowed to age over night at ambient temperature. Finally, 1 part concentrate is mixed with 5 parts carbonated water, filled into bottles and sealed.

### Application Example 9

### Drink Chocolate Instant Powder

### Preparation A: Standard Preparation

### Preparation B-D: Preparation According to the Present Invention

| Ingredient | Use in weight % | | | | | | |
|---|---|---|---|---|---|---|---|
| Preparation | A | B | C | D | E | F | G |
| Sucrose, extra fine | 73.50 | 48.48 | 73.69 | 13.33 | 48.48 | 73.69 | 13.33 |
| Cocoa powder alka- | 25.00 | 25.00 | 25.00 | 25.00 | 25.00 | 25.00 | 25.00 |
| lized, 10-12% by | | | | | | | |
| weight fat | | | | | | | |
| Maltodextrin DE15- | 0.43 | 0.43 | 0.43 | 0.43 | 0.43 | 0.43 | 0.43 |
| 19 from corn starch | | | | | | | |
| Salt (NaCl), extra | 0.43 | 0.43 | 0.43 | 0.43 | 0.43 | 0.43 | 0.43 |
| fine | | | | | | | |
| Ascorbic acid | 0.29 | 0.29 | 0.29 | 0.29 | 0.29 | 0.29 | 0.29 |
| Vitamin mix M8 40- | 0.145 | 0.145 | 0.145 | 0.145 | 0.145 | 0.145 | 0.145 |
| 1507 | | | | | | | |
| D-Allulose, crystal- | — | 25.00 | — | 60.00 | 25.00 | — | 60.00 |
| line | | | | | | | |
| 6″-O-Butanoyl-HG, | — | 0.0050 | 0.0040 | 0.0045 | | | |
| compound (41) | | | | | | | |
| 6″-O-Pentanoyl- | — | | | | 0.0030 | 0.0024 | 0.0027 |
| HG, compound (73) | | | | | | | |
| Hesperetin dihydro- | — | — | 0.01 | 0.01 | — | 0.01 | 0.01 |
| chalcone (I) | | | | | | | |
| Hesperetin | — | 0.05 | — | — | 0.05 | — | — |
| Rebaudioside A | — | — | — | 0.02 | — | — | 0.02 |
| Phloretin | — | 0.03 | — | — | 0.03 | — | — |
| Rebaudioside M | — | — | — | 0.30 | — | — | 0.30 |
| Phyllodulcin (70%, | — | 0.01 | — | 0.01 | 0.01 | — | 0.01 |
| dried Oamacha ex- | | | | | | | |
| tract) | | | | | | | |

Standard dosage in milk for preparing an choco beverage: 6.9% by weight.

### Application Example 10

### Instant Ice Tea Type Peach

### Preparation A: Standard Preparation

### Preparation B-D: Preparation According to the Present Invention

| Ingredient | Use in weight % | | | | | | |
|---|---|---|---|---|---|---|---|
| Preparation | A | B | C | D | E | F | G |
| Sucrose, extra fine | 95.06 | 70.09 | 70.25 | — | 70.09 | 70.25 | — |
| Citric acid, anhy- | 2.20 | 2.00 | 2.00 | 1.80 | 2.00 | 2.00 | 1.80 |
| drous | | | | | | | |
| Black Tea extract, | 1.34 | 1.34 | 1.34 | 1.34 | 1.34 | 1.34 | 1.34 |
| powdered | | | | | | | |
| Peach aroma spray | 0.66 | 0.66 | 0.66 | 0.66 | 0.66 | 0.66 | 0.66 |
| dried | | | | | | | |
| Tea aroma type | 0.27 | 0.27 | 0.27 | 0.27 | 0.27 | 0.27 | 0.27 |
| Ceylon dried | | | | | | | |
| Trisodium citrate | 0.25 | 0.25 | 0.25 | 0.25 | 0.25 | 0.25 | 0.25 |
| Ascorbic acid | 0.22 | 0.22 | 0.22 | 0.22 | 0.22 | 0.22 | 0.22 |
| Polydextrose | — | — | — | 25.00 | — | — | 25.00 |
| D-Allulose, crystal- | — | 25.00 | 25.00 | 70.00 | 25.00 | 25.00 | 70.00 |
| line | | | | | | | |
| 6″-O-Butanoyl-HG, | — | 0.0057 | 0.0046 | 0.0051 | — | — | — |
| compound (41) | | | | | | | |
| 6″-O-Pentanoyl- | — | — | — | — | 0.0034 | 0.0027 | 0.0031 |
| HG, compound (73) | | | | | | | |
| Phyllodulcin (80%, | — | — | 0.01 | 0.01 | — | 0.01 | 0.01 |
| dried Oamacha ex- | | | | | | | |
| tract) | | | | | | | |
| Hesperetin | — | 0.05 | — | — | 0.05 | — | — |
| Rebaudioside A | — | — | — | 0.01 | — | — | 0.01 |
| Phloretin | — | 0.02 | — | — | 0.02 | — | — |
| Rebaudioside M | — | — | — | 0.20 | — | — | 0.20 |
| Homoeriodictyol- | — | 0.10 | — | 0.05 | 0.10 | — | 0.05 |
| Sodium salt | | | | | | | |

Standard dosage in water for preparing a peach ice tea beverage: 7.5% by weight.

### Application Example 11

### Ice Tea

### Comparative Example with Sucrose (A)

| | Preparation (wt.- % or ppm if specified accordingly) | | | | | | | | | | | |
|---|---|---|---|---|---|---|---|---|---|---|---|---|
| Ingrediens | A | B | C | D | E | F | G | H | I | J | K | L |
| sucrose | 7.00 | 3.50 | 3.50 | 3.50 | 3.50 | 3.50 | 3.50 | 3.50 | 3.50 | 3.50 | 3.50 | 3.50 |
| D-allulose | — | 2.00 | — | 2.00 | — | 2.00 | 2.00 | — | 2.00 | — | 2.00 | 2.00 |
| 6″-O- | — | 4 | 2.5 | 1.75 | 1.75 | 2.5 | — | — | — | — | — | 2.5 |
| Butanoyl- | | ppm | ppm | ppm | ppm | ppm | | | | | | ppm |
| HG. | | | | | | | | | | | | |
| compound | | | | | | | | | | | | |
| (41) | | | | | | | | | | | | |
| 6″-O- | — | — | — | — | — | — | 2.5 | 1.75 | 1 | 1 | 1.4 | 1.4 |
| Pentanoyl- | | | | | | | ppm | ppm | ppm | ppm | ppm | ppm |
| HG. | | | | | | | | | | | | |
| compound | | | | | | | | | | | | |
| (73) | | | | | | | | | | | | |
| Hesperetin | — | — | 5 | — | — | — | — | 5 | — | — | — | — |
| (HT) | | | ppm | | | | | ppm | | | | |
| Phloretin | — | — | — | 15 | — | — | — | — | 15 | — | — | — |
| (PH) | | | | ppm | | | | | ppm | | | |
| Hesperetin | — | — | — | — | 15 | — | — | — | — | 15 | — | — |
| dihydrochal- | | | | | ppm | | | | | ppm | | |
| cone (HC) | | | | | | | | | | | | |
| Alpha- | — | — | — | — | — | 10 | — | — | — | — | 10 | — |
| glycosylated | | | | | | ppm | | | | | ppm | |
| rubusosides | | | | | | | | | | | | |
| (RS) | | | | | | | | | | | | |
| Phyllodulcin | — | — | — | — | — | — | — | — | — | — | — | — |
| (75%. as | | | | | | | | | | | | |
| dried | | | | | | | | | | | | |
| Oamacha | | | | | | | | | | | | |
| extract) | | | | | | | | | | | | |
| Citric acid | 0.15 | 0.15 | 0.15 | 0.15 | 0.15 | 0.15 | 0.15 | 0.15 | 0.15 | 0.15 | 0.15 | 0.15 |
| ascorbic | 0.02 | 0.02 | 0.02 | 0.02 | 0.02 | 0.02 | 0.02 | 0.02 | 0.02 | 0.02 | 0.02 | 0.02 |
| acid | | | | | | | | | | | | |
| Black tea | 0.15 | 0.15 | 0.15 | 0.15 | 0.15 | 0.15 | 0.15 | 0.15 | 0.15 | 0.15 | 0.15 | 0.15 |
| extrakt | | | | | | | | | | | | |
| natural | 0.10 | 0.10 | 0.10 | 0.10 | 0.10 | 0.10 | 0.10 | 0.10 | 0.10 | 0.10 | 0.10 | 0.10 |
| lemon | | | | | | | | | | | | |
| flavor | | | | | | | | | | | | |

| | |
|---|---|
| water | to 100% |

The ingredients were mixed in the order listed in the order listed into bottled and sterilized.

1-14. (canceled)

15. A method of modulating sweet taste of one or more sweet tasting substances comprising:
(a) contacting the one or more sweet tasting substances with one or more compounds of formula (I): 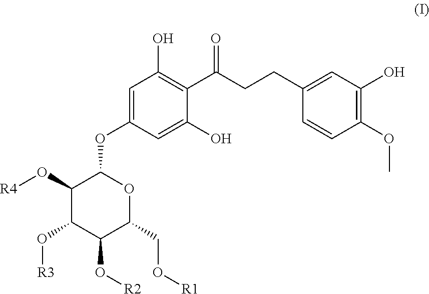
wherein R1, R2, R3, and R4 are independently selected from hydrogen, acetyl, propionyl, butanoyl, pentanoyl, hexanoyl, heptanoyl, octanoyl, nonanoyl, and decanoyl, provided that at least one of R1, R2, R3, and R4 is other than hydrogen; and
(b) thereby, modulating the sweet taste of the one or more sweet tasting substances.

16. The method of claim 15, wherein the one or more compounds of formula (I) are selected from:

| Compound | R1 | R2 | R3 | R4 |
|---|---|---|---|---|
| 2 | H | H | H | acetyl |
| 3 | H | H | acetyl | H |
| 4 | H | H | acetyl | acetyl |
| 5 | H | acetyl | H | H |
| 6 | H | acetyl | H | acetyl |
| 7 | H | acetyl | acetyl | H |
| 8 | H | acetyl | acetyl | acetyl |
| 9 | acetyl | H | H | H |
| 10 | acetyl | H | H | acetyl |
| 11 | acetyl | H | acetyl | H |
| 12 | acetyl | H | acetyl | acetyl |
| 13 | acetyl | acetyl | H | H |
| 14 | acetyl | acetyl | H | acetyl |
| 15 | acetyl | acetyl | acetyl | H |
| 16 | acetyl | acetyl | acetyl | acetyl |
| 18 | H | H | H | propionyl |
| 19 | H | H | propionyl | H |
| 20 | H | H | propionyl | propionyl |
| 21 | H | propionyl | H | H |
| 22 | H | propionyl | H | propionyl |
| 23 | H | propionyl | propionyl | H |
| 24 | H | propionyl | propionyl | propionyl |
| 25 | propionyl | H | H | H |
| 26 | propionyl | H | H | propionyl |
| 27 | propionyl | H | propionyl | H |
| 28 | propionyl | H | propionyl | propionyl |
| 29 | propionyl | propionyl | H | H |
| 30 | propionyl | propionyl | H | propionyl |
| 31 | propionyl | propionyl | propionyl | H |
| 32 | propionyl | propionyl | propionyl | propionyl |
| 34 | H | H | H | butanoyl |
| 35 | H | H | butanoyl | H |
| 36 | H | H | butanoyl | butanoyl |
| 37 | H | butanoyl | H | H |
| 38 | H | butanoyl | H | butanoyl |
| 39 | H | butanoyl | butanoyl | H |
| 40 | H | butanoyl | butanoyl | butanoyl |
| 41 | butanoyl | H | H | H |
| 42 | butanoyl | H | H | butanoyl |
| 43 | butanoyl | H | butanoyl | H |
| 44 | butanoyl | H | butanoyl | butanoyl |
| 45 | butanoyl | butanoyl | H | H |
| 46 | butanoyl | butanoyl | H | butanoyl |
| 47 | butanoyl | butanoyl | butanoyl | H |
| 48 | butanoyl | butanoyl | butanoyl | butanoyl |
| 50 | H | H | H | pentanoyl |
| 51 | H | H | pentanoyl | H |
| 52 | H | H | pentanoyl | pentanoyl |
| 53 | H | pentanoyl | H | H |
| 54 | H | pentanoyl | H | pentanoyl |
| 55 | H | pentanoyl | pentanoyl | H |
| 56 | H | pentanoyl | pentanoyl | pentanoyl |
| 57 | pentanoyl | H | H | H |
| 58 | pentanoyl | H | H | pentanoyl |
| 59 | pentanoyl | H | pentanoyl | H |
| 60 | pentanoyl | H | pentanoyl | pentanoyl |
| 61 | pentanoyl | pentanoyl | H | H |
| 62 | pentanoyl | pentanoyl | H | pentanoyl |
| 63 | pentanoyl | pentanoyl | pentanoyl | H |
| 64 | pentanoyl | pentanoyl | pentanoyl | pentanoyl |
| 66 | H | H | H | hexanoyl |
| 67 | H | H | hexanoyl | H |
| 68 | H | H | hexanoyl | hexanoyl |
| 69 | H | hexanoyl | H | H |
| 70 | H | hexanoyl | H | hexanoyl |
| 71 | H | hexanoyl | hexanoyl | H |
| 72 | H | hexanoyl | hexanoyl | hexanoyl |
| 73 | hexanoyl | H | H | H |
| 74 | hexanoyl | H | H | hexanoyl |
| 75 | hexanoyl | H | hexanoyl | H |
| 76 | hexanoyl | H | hexanoyl | hexanoyl |
| 77 | hexanoyl | hexanoyl | H | H |
| 78 | hexanoyl | hexanoyl | H | hexanoyl |
| 79 | hexanoyl | hexanoyl | hexanoyl | H |
| 80 | hexanoyl | hexanoyl | hexanoyl | hexanoyl |
| 82 | H | H | H | heptanoyl |
| 83 | H | H | heptanoyl | H |
| 84 | H | H | heptanoyl | heptanoyl |
| 85 | H | heptanoyl | H | H |
| 86 | H | heptanoyl | H | heptanoyl |
| 87 | H | heptanoyl | heptanoyl | H |
| 88 | H | heptanoyl | heptanoyl | heptanoyl |
| 89 | heptanoyl | H | H | H |
| 90 | heptanoyl | H | H | heptanoyl |
| 91 | heptanoyl | H | heptanoyl | H |
| 92 | heptanoyl | H | heptanoyl | heptanoyl |
| 93 | heptanoyl | heptanoyl | H | H |
| 94 | heptanoyl | heptanoyl | H | heptanoyl |
| 95 | heptanoyl | heptanoyl | heptanoyl | H |
| 96 | heptanoyl | heptanoyl | heptanoyl | heptanoyl |
| 98 | H | H | H | octanoyl |
| 99 | H | H | octanoyl | H |
| 100 | H | H | octanoyl | octanoyl |
| 101 | H | octanoyl | H | H |
| 102 | H | octanoyl | H | octanoyl |
| 103 | H | octanoyl | octanoyl | H |
| 104 | H | octanoyl | octanoyl | octanoyl |
| 105 | octanoyl | H | H | H |
| 106 | octanoyl | H | H | octanoyl |
| 107 | octanoyl | H | octanoyl | H |
| 108 | octanoyl | H | octanoyl | octanoyl |
| 109 | octanoyl | octanoyl | H | H |
| 110 | octanoyl | octanoyl | H | octanoyl |
| 111 | octanoyl | octanoyl | octanoyl | H |
| 112 | octanoyl | octanoyl | octanoyl | octanoyl |
| 114 | H | H | H | nonanoyl |
| 115 | H | H | nonanoyl | H |
| 116 | H | H | nonanoyl | nonanoyl |
| 117 | H | nonanoyl | H | H |
| 118 | H | nonanoyl | H | nonanoyl |
| 119 | H | nonanoyl | nonanoyl | H |
| 120 | H | nonanoyl | nonanoyl | nonanoyl |
| 121 | nonanoyl | H | H | H |
| 122 | nonanoyl | H | H | nonanoyl |
| 123 | nonanoyl | H | nonanoyl | H |
| 124 | nonanoyl | H | nonanoyl | nonanoyl |
| 125 | nonanoyl | nonanoyl | H | H |
| 126 | nonanoyl | nonanoyl | H | nonanoyl |
| 127 | nonanoyl | nonanoyl | nonanoyl | H |
| 128 | nonanoyl | nonanoyl | nonanoyl | nonanoyl |
| 130 | H | H | H | decanoyl |
| 131 | H | H | decanoyl | H |
| 132 | H | H | decanoyl | decanoyl |
| 133 | H | decanoyl | H | H |
| 134 | H | decanoyl | H | decanoyl |
| 135 | H | decanoyl | decanoyl | H |
| 136 | H | decanoyl | decanoyl | decanoyl |
| 137 | decanoyl | H | H | H |
| 138 | decanoyl | H | H | decanoyl |
| 139 | decanoyl | H | decanoyl | H |
| 140 | decanoyl | H | decanoyl | decanoyl |
| 141 | decanoyl | decanoyl | H | H |
| 142 | decanoyl | decanoyl | H | decanoyl |
| 143 | decanoyl | decanoyl | decanoyl | H |
| 144 | decanoyl | decanoyl | decanoyl | decanoyl |

17. The method of claim 15, wherein R2, R3, and R4 are each hydrogen.

18. The method of claim 15, wherein the one or more compounds of formula (I) are selected from:

| Compound | Compound | Structure |
|---|---|---|
| 41 | 6″-O-Butanoyl-HG | 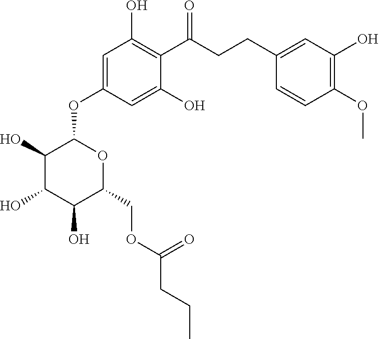 [SMILES: CCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 57 | 6″-O-Pentanoyl-HG | 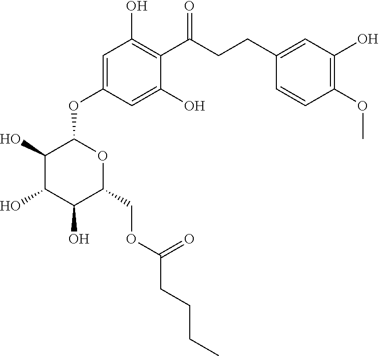 [SMILES: CCCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 73 | 6″-O-Hexanoyl-HG | 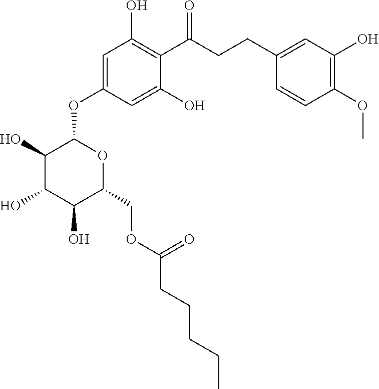 [SMILES: CCCCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 89 | 6″-O-Heptanoyl-HG | 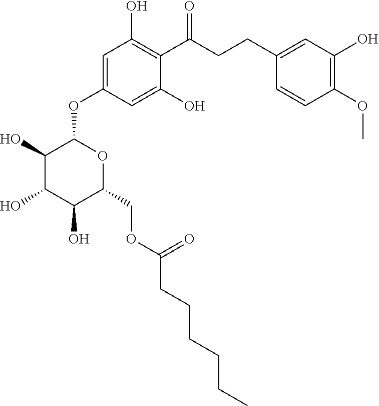 [SMILES: CCCCCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 25 | 6″-O-Propanoyl-HG | 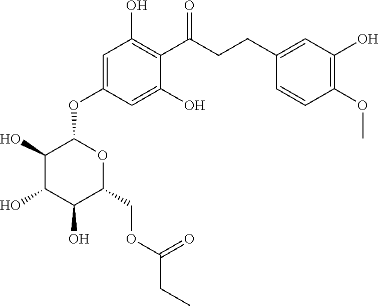 [SMILES: CCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 9 | 6″-O-Acetyl-HG | 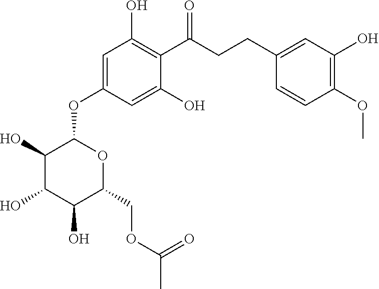 [SMILES: COc1ccc(CCC(=O)c2c(O)cc(O[C@@H]3O[C@H](COC(C)=O)[C@@H](O)[C@H](O)[C@H]3O)cc2O)cc1O] |
| 105 | 6″-O-Octanoyl-HG | 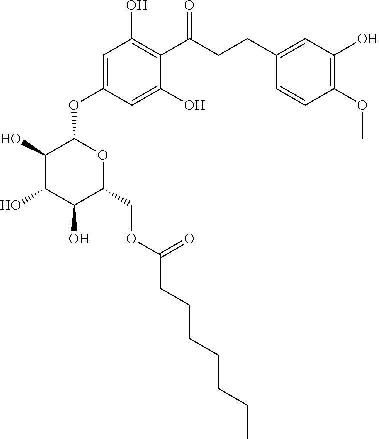 [SMILES: CCCCCCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 121 | 6″-O-Nonanoyl-HG | 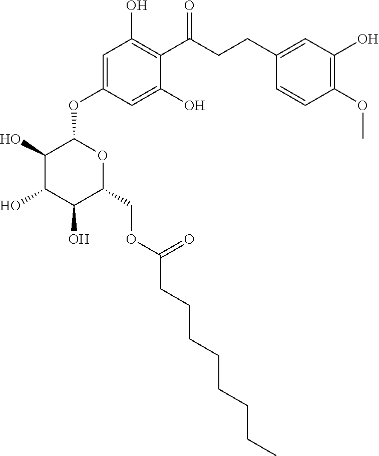 [SMILES: CCCCCCCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 137 | 6″-O-Decanoyl-HG | 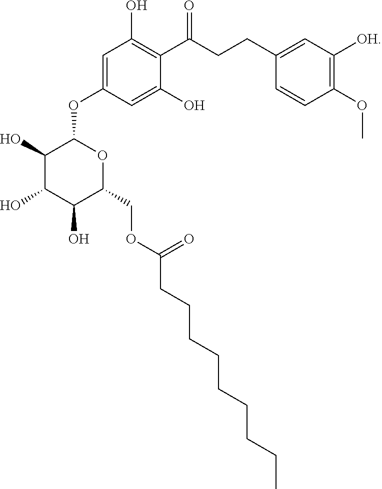 [SMILES: CCCCCCCCCC(=O)OC[C@H]1O[C@@H](Oc2cc(O)c(C(=O)CCc3ccc(OC)c(O)c3)c(O)c2)[C@H](O)[C@@H](O)[C@@H]1O] |

19. The method of claim 15, wherein sweet tasting substances are selected from natural sweet tasting substances and synthetic sweet tasting substances.

20. A compound of formula (I) 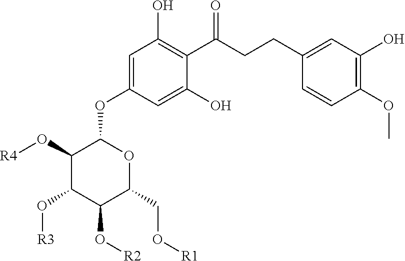
wherein the compound is selected from:

| Compound | R1 | R2 | R3 | R4 |
|---|---|---|---|---|
| 2 | H | H | H | acetyl |
| 3 | H | H | acetyl | H |
| 4 | H | H | acetyl | acetyl |
| 5 | H | acetyl | H | H |
| 6 | H | acetyl | H | acetyl |
| 7 | H | acetyl | acetyl | H |
| 8 | H | acetyl | acetyl | acetyl |
| 9 | acetyl | H | H | H |
| 10 | acetyl | H | H | acetyl |
| 11 | acetyl | H | acetyl | H |
| 12 | acetyl | H | acetyl | acetyl |
| 13 | acetyl | acetyl | H | H |
| 14 | acetyl | acetyl | H | acetyl |
| 15 | acetyl | acetyl | acetyl | H |
| 16 | acetyl | acetyl | acetyl | acetyl |
| 18 | H | H | H | propionyl |
| 19 | H | H | propionyl | H |
| 20 | H | H | propionyl | propionyl |
| 21 | H | propionyl | H | H |
| 22 | H | propionyl | H | propionyl |
| 23 | H | propionyl | propionyl | H |
| 24 | H | propionyl | propionyl | propionyl |
| 25 | propionyl | H | H | H |
| 26 | propionyl | H | H | propionyl |
| 27 | propionyl | H | propionyl | H |
| 28 | propionyl | H | propionyl | propionyl |
| 29 | propionyl | propionyl | H | H |
| 30 | propionyl | propionyl | H | propionyl |
| 31 | propionyl | propionyl | propionyl | H |
| 32 | propionyl | propionyl | propionyl | propionyl |
| 34 | H | H | H | butanoyl |
| 35 | H | H | butanoyl | H |
| 36 | H | H | butanoyl | butanoyl |
| 37 | H | butanoyl | H | H |
| 38 | H | butanoyl | H | butanoyl |
| 39 | H | butanoyl | butanoyl | H |
| 40 | H | butanoyl | butanoyl | butanoyl |
| 41 | butanoyl | H | H | H |
| 42 | butanoyl | H | H | butanoyl |
| 43 | butanoyl | H | butanoyl | H |
| 44 | butanoyl | H | butanoyl | butanoyl |
| 45 | butanoyl | butanoyl | H | H |
| 46 | butanoyl | butanoyl | H | butanoyl |
| 47 | butanoyl | butanoyl | butanoyl | H |
| 48 | butanoyl | butanoyl | butanoyl | butanoyl |
| 50 | H | H | H | pentanoyl |
| 51 | H | H | pentanoyl | H |
| 52 | H | H | pentanoyl | pentanoyl |
| 53 | H | pentanoyl | H | H |
| 54 | H | pentanoyl | H | pentanoyl |
| 55 | H | pentanoyl | pentanoyl | H |
| 56 | H | pentanoyl | pentanoyl | pentanoyl |
| 57 | pentanoyl | H | H | H |
| 58 | pentanoyl | H | H | pentanoyl |
| 59 | pentanoyl | H | pentanoyl | H |
| 60 | pentanoyl | H | pentanoyl | pentanoyl |
| 61 | pentanoyl | pentanoyl | H | H |
| 62 | pentanoyl | pentanoyl | H | pentanoyl |
| 63 | pentanoyl | pentanoyl | pentanoyl | H |
| 64 | pentanoyl | pentanoyl | pentanoyl | pentanoyl |
| 66 | H | H | H | hexanoyl |
| 67 | H | H | hexanoyl | H |
| 68 | H | H | hexanoyl | hexanoyl |
| 69 | H | hexanoyl | H | H |
| 70 | H | hexanoyl | H | hexanoyl |
| 71 | H | hexanoyl | hexanoyl | H |
| 72 | H | hexanoyl | hexanoyl | hexanoyl |
| 73 | hexanoyl | H | H | H |
| 74 | hexanoyl | H | H | hexanoyl |
| 75 | hexanoyl | H | hexanoyl | H |
| 76 | hexanoyl | H | hexanoyl | hexanoyl |
| 77 | hexanoyl | hexanoyl | H | H |
| 78 | hexanoyl | hexanoyl | H | hexanoyl |
| 79 | hexanoyl | hexanoyl | hexanoyl | H |
| 80 | hexanoyl | hexanoyl | hexanoyl | hexanoyl |
| 82 | H | H | H | heptanoyl |
| 83 | H | H | heptanoyl | H |
| 84 | H | H | heptanoyl | heptanoyl |
| 85 | H | heptanoyl | H | H |
| 86 | H | heptanoyl | H | heptanoyl |
| 87 | H | heptanoyl | heptanoyl | H |
| 88 | H | heptanoyl | heptanoyl | heptanoyl |
| 89 | heptanoyl | H | H | H |
| 90 | heptanoyl | H | H | heptanoyl |
| 91 | heptanoyl | H | heptanoyl | H |
| 92 | heptanoyl | H | heptanoyl | heptanoyl |
| 93 | heptanoyl | heptanoyl | H | H |
| 94 | heptanoyl | heptanoyl | H | heptanoyl |
| 95 | heptanoyl | heptanoyl | heptanoyl | H |
| 96 | heptanoyl | heptanoyl | heptanoyl | heptanoyl |
| 98 | H | H | H | octanoyl |
| 99 | H | H | octanoyl | H |
| 100 | H | H | octanoyl | octanoyl |
| 101 | H | octanoyl | H | H |
| 102 | H | octanoyl | H | octanoyl |
| 103 | H | octanoyl | octanoyl | H |
| 104 | H | octanoyl | octanoyl | octanoyl |
| 105 | octanoyl | H | H | H |
| 106 | octanoyl | H | H | octanoyl |
| 107 | octanoyl | H | octanoyl | H |
| 108 | octanoyl | H | octanoyl | octanoyl |
| 109 | octanoyl | octanoyl | H | H |
| 110 | octanoyl | octanoyl | H | octanoyl |
| 111 | octanoyl | octanoyl | octanoyl | H |
| 112 | octanoyl | octanoyl | octanoyl | octanoyl |
| 114 | H | H | H | nonanoyl |
| 115 | H | H | nonanoyl | H |
| 116 | H | H | nonanoyl | nonanoyl |
| 117 | H | nonanoyl | H | H |
| 118 | H | nonanoyl | H | nonanoyl |
| 119 | H | nonanoyl | nonanoyl | H |
| 120 | H | nonanoyl | nonanoyl | nonanoyl |
| 121 | nonanoyl | H | H | H |
| 122 | nonanoyl | H | H | nonanoyl |
| 123 | nonanoyl | H | nonanoyl | H |
| 124 | nonanoyl | H | nonanoyl | nonanoyl |
| 125 | nonanoyl | nonanoyl | H | H |
| 126 | nonanoyl | nonanoyl | H | nonanoyl |
| 127 | nonanoyl | nonanoyl | nonanoyl | H |
| 128 | nonanoyl | nonanoyl | nonanoyl | nonanoyl |
| 130 | H | H | H | decanoyl |
| 131 | H | H | decanoyl | H |
| 132 | H | H | decanoyl | decanoyl |
| 133 | H | decanoyl | H | H |
| 134 | H | decanoyl | H | decanoyl |
| 135 | H | decanoyl | decanoyl | H |
| 136 | H | decanoyl | decanoyl | decanoyl |
| 137 | decanoyl | H | H | H |
| 138 | decanoyl | H | H | decanoyl |
| 139 | decanoyl | H | decanoyl | H |
| 140 | decanoyl | H | decanoyl | decanoyl |
| 141 | decanoyl | decanoyl | H | H |
| 142 | decanoyl | decanoyl | H | decanoyl |
| 143 | decanoyl | decanoyl | decanoyl | H |
| 144 | decanoyl | decanoyl | decanoyl | decanoyl |

21. The compound of claim 20, wherein R2, R3, and R4 are each hydrogen.

22. The compound of claim 20 selected from:

| Compound | Compound | Structure |
|---|---|---|
| 41 | 6″-O-Butanoyl-HG | 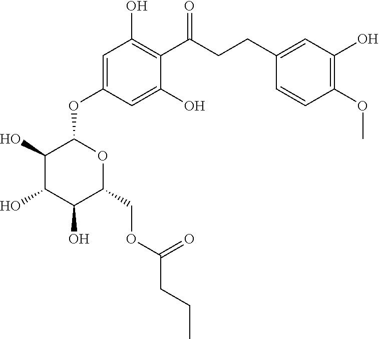 [SMILES: CCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 57 | 6″-O-Pentanoyl-HG | 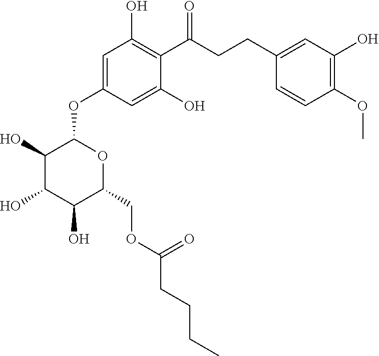 [SMILES: CCCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 73 | 6″-O-Hexanoyl-HG | 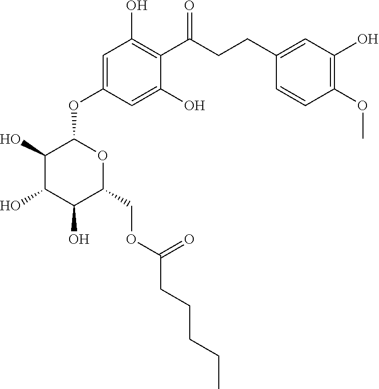 [SMILES: CCCCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 89 | 6″-O-Heptanoyl-HG | 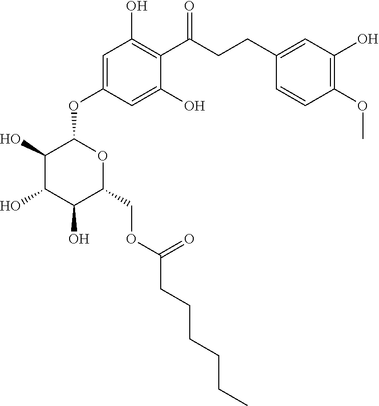 [SMILES: CCCCCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 25 | 6″-O-Propanoyl-HG | 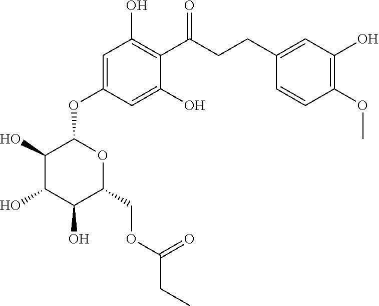 [SMILES: CCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 9 | 6″-O-Acetyl-HG | 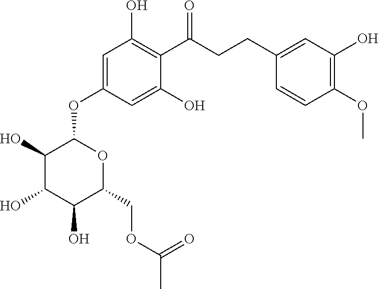 [SMILES: COc1ccc(CCC(=O)c2c(O)cc(O[C@@H]3O[C@H](COC(C)=O)[C@@H](O)[C@H](O)[C@H]3O)cc2O)cc1O] |
| 105 | 6″-O-Octanoyl-HG | 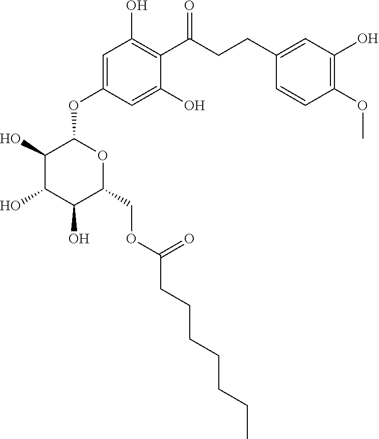 [SMILES: CCCCCCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 121 | 6″-O-Nonanoyl-HG | 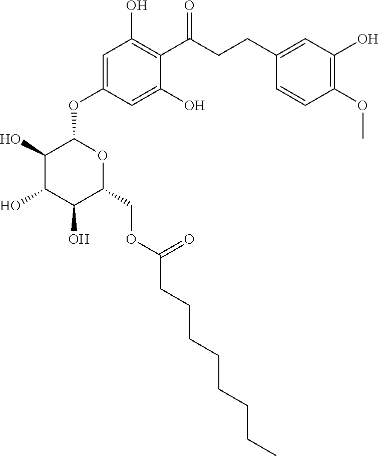 [SMILES: CCCCCCCCC(=O)OC[C@H]1O[C@@H](Oc2cc(O)c(C(=O)CCc3ccc(OC)c(O)c3)c(O)c2)[C@H](O)[C@@H](O)[C@@H]1O] |
| 137 | 6″-O-Decanoyl-HG | 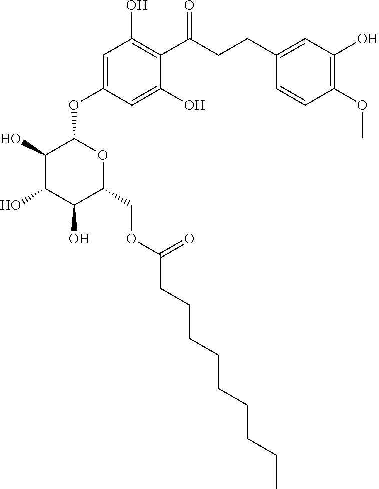 [SMILES: CCCCCCCCCC(=O)OC[C@H]1O[C@@H](Oc2cc(O)c(C(=O)CCc3ccc(OC)c(O)c3)c(O)c2)[C@H](O)[C@@H](O)[C@@H]1O] |

23. A composition comprising the compound of claim 20 and one or more sweet tasting substances selected from natural sweet tasting substance(s) and synthetic sweet tasting substances.

24. A method for producing the compound of formula (I) of claim 15 comprising:
(i) providing one or more precursor compounds selected from hesperetin dihydrochalcone, hesperidin dihydrochalcone, and hesperetin dihydrochalcone-4′-O-β-d-glucoside (HG);
(ii) providing one or more acyl donors; and
(iii) contacting the one or more precursor compounds of step (i) with the one or more acyl donors of step (ii) under conditions sufficient to effect a chemical or enzymatic acylation.

25. The method of claim 24, wherein the one or more acyl donors are selected from ethanoyl donors, propanoyl donors, butanoyl donors, pentanoyl donors, hexanoyl donors, heptanoyl donors, octanoyl donors, nonanoyl donors, and decanoyl donors.

26. The method of claim 25, wherein the one or more acyl donors are selected from triacetin, acetic acid, methyl acetate, ethyl acetate, acetic anhydride, tripropionin, propionic acid, methyl propanoate, ethyl propanoate, tributyrin, butanoic acid, methyl butanoate, ethyl butanoate, tripentanoin, pentanoic acid, methyl pentanoate, ethyl pentanoate, trihexanoin, hexanoic acid, methyl hexanoate, ethyl hexanoate, triheptanoin, heptanoic acid, methyl heptanoate, ethyl heptanoate, trioctanoin, octanoic acid, methyl octanoate, ethyl octanoate, trinonanoin, nonanoic acid, methyl nonanoate, ethyl nonanoate, tricaprin, decanoic acid, methyl decanoate, and ethyl decanoate.

27. The method of claim 24, wherein step (iii) comprises an enzymatic acylation carried out with a lipase.

28. The method of claim 27, wherein the lipase is obtained from a microorganism selected from Candida antarctica A, Candida antarctica B, Candida rugosa, Burkholderia cepacia, Rhizopus sp., Rhizomucor miehei, Mucor javanicus, Yarrowia lipolytica, Geotrichum candidum, Aspergillus niger, Aspergillus oryzae, Pseudomonas alcaligenes, Pseudomonas mendocina, Thermomyces lanuginosus, and Chromobacterium viscosum.

29. The method of claim 24, wherein the one or more precursor compounds of step (i) are present in a plant extract.

30. The method of claim 29, wherein the plant extract is an extract of Balanophora harlandii.
